(19) United States
(12) Patent Application Publication
(10) Pub. No.: US 2024/0174598 A1
(43) Pub. Date: May 30, 2024

(54) COMPOUNDS FOR THE TREATMENT OF DISORDERS AND SALTS AND POLYMORPHS THEREOF

(71) Applicant: OccuRx Pty Ltd, Melbourne (AU)

(72) Inventors: Darren James Kelly, Melbourne (AU); Michelle Papadimitriou, Melbourne (AU); Chris Bums, Melbourne (AU); Eric Daniels, Melbourne (AU)

(21) Appl. No.: 18/281,716

(22) PCT Filed: Mar. 15, 2022
(86) PCT No.: PCT/US2022/020351

§ 371 Date: Sep. 12, 2023

Related U.S. Application Data
(60) Provisional application No. 63/316,652, filed on Mar. 4, 2022.
(60) Provisional application No. 63/162,425, filed on Mar. 17, 2021.

(51) Int. Cl. C07C 235/38; A61K 31/167; C07C 211/07; C07C 229/26

(52) U.S. Cl. CPC C07C 235/38; A61K 31/167; C07C 211/07; C07C 229/26; C07B 2200/13

(57) ABSTRACT

Disclosed herein are salts of the compound of Formula (I) and polymorphs thereof, methods of making the same, and their methods of use in treating eye diseases and disorders, particularly those associated with inflammation and/or vascular proliferation in subjects. The methods include administering therapeutically effective amounts of salts of the compound of Formula (I) and polymorphic forms thereof, to a subject in need thereof.

### BACKGROUND

### Field of the Disclosure

The present application relates to the fields of pharmaceutical chemistry, formulation science, and medicine. In particular, it relates to salt forms of the compound of Formula (I), polymorphs thereof, and methods of making and using such salts forms and polymorphs for treating eye diseases and disorders, particularly eye diseases and disorders associated with inflammation and/or vascular proliferation. The present disclosure also relates to using the salt forms described herein for the treatment of diseases or disorders associated with fibrosis.

### Description of the Related Art

Ocular diseases that involve inflammation and/or vascular proliferation usually, but not always, relate to the anterior- and posterior-segments of the eye. For example, ocular disorders that have an etiology in inflammation and/or vascular proliferation could be corneal edema, anterior and posterior uveitis, pterygium, corneal diseases that are caused by infections from microbes or microorganisms such as bacteria, viruses, fungi, amoebas and parasites, dry eye, conjunctivitis, allergy- and laser-induced exudation, non-age related macular degeneration, macular edema, diabetic retinopathy (DR), age-related macular degeneration (Kim et al. 2001; A. M. Joussen et al. 2004; S. C. Pflugfelder 2004), ocular von Hippel-Lindau disease, which is characterised by fine vascular proliferation in the retina, and proliferative vitreoretinopathy (PVR).

One of the above-mentioned ocular diseases, DR, is a common complication of diabetes and remains one of the leading causes of vision loss. See Cheung et al. 2005; Santos et al. 2005. Vision loss in DR develops by slow and progressive alterations to the retinal microvasculature (pericytes and endothelial cells) leading to breakdown of the blood-retinal barrier, pathological angiogenesis and scarring. Based on the extent of vascular abnormalities, DR can be broadly categorized into non-proliferative DR (NPDR) and proliferative DR (PDR). See Klein et al. 2004. In cases of NPDR, hyperglycaemia induces thickening of capillary basement membrane, apoptosis or ‘dropout’ of pericytes, microaneurysms and vascular leakage. Blockade of retinal capillaries causes localized hypoxia, which increases the production of angiogenic growth factors. In some microvessels, endothelial cells become apoptotic resulting in acellular capillaries (devoid of both pericytes and endothelial cells), capillary closure and areas of retinal non-perfusion. Adherent leukocytes may also contribute to the lesion by causing retinal capillary occlusion. See Joussen, et al. 2004. Multiple haemorrhages, soft exudates, cotton wool spots, intraretinal microvascular abnormalities and venous beading and loops develop. Increased areas of tissue non-perfusion stimulate the production of angiogenic factors leading to the proliferation of vessels, which is the hallmark feature of PDR. Retinal angiogenesis can be accompanied by fibrosis resulting in a fibrovascular ridge, which extends into the vitreous cavity or on the surface of the retina. Contraction of the fibrovascular ridge causes retinal detachment and vision loss and blindness. See Watkins 2003.

The pathogenesis of DR is not fully understood. However, metabolic and biochemical changes, such as increased flux of glucose through the polyol pathway, activation of protein kinase C, oxidative damage and increased advanced glycation end product formations are contributors in the development of DR. See Cheung et al. 2005. Accumulating evidence indicate that vascular endothelial growth factor (VEGF) plays a critical role in angiogenesis in DR, see Sarlos, et al. 2003, while intercellular adhesion molecule (ICAM-1) mediated leukocytosis resulting in secondary endothelial damage. See Joussen, et al. 2002; Khalfaoui, et al. 2009. Recently, DR has also been recognized as a chronic inflammatory disease. See Adamis 2002; Joussen, et al. 2004. With this notion, studies demonstrated that anti-inflammatory therapy prevents classic histopathological features of DR: acellular capillary formation, retinal haemorrhage development, microaneurysm progress, and pericyte loss. See Adamis 2002; Joussen, Poulaki et al. 2002.

The current DR is treated by laser photocoagulation, a procedure that destroys angiogenic vessels and the surrounding hypoxic tissue. See Aiello 2003. Although beneficial, laser photocoagulation can destroy healthy retina, and the disease continues despite intensive treatment. Therefore, less invasive therapies are being investigated, with a particular focus on the inhibition of injurious molecules such as VEGF and ICAM-1. See Arita, et al.; Sarlos, et al. 2003; Khalfaoui, et al. 2009. There remains a need for further therapies for treating eye diseases associated with inflammation and/or vascular proliferation such as diabetic retinopathy as well as corneal edema, anterior and posterior uveitis, pterygium, corneal diseases that are caused by infections from microbes or microorganisms such as bacteria, viruses, fungi, amoebas and parasites, dry eye, conjunctivitis, allergy- and laser-induced exudation, non-age related macular degeneration, macular edema, age-related macular degeneration and ocular von Hippel-Lindau disease.

### SUMMARY

In one aspect, the present application is directed to pharmaceutically acceptable salts of the compound of Formula (I), including the crystalline forms thereof.

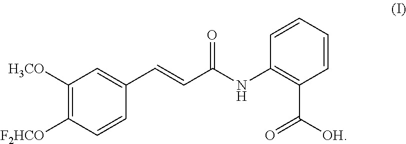 [SMILES: COc1cc(/C=C/C(=O)Nc2ccccc2C(=O)O)ccc1OC(F)F]

In some embodiments of this aspect of the disclosure, the pharmaceutically acceptable salt of the compound of Formula (I) may be a sodium salt, a potassium salt, a zinc salt, a magnesium salt, or a calcium salt of the compound of Formula (I).

In some embodiments of the first aspect of the disclosure, the pharmaceutically acceptable salt of the compound of Formula (I) may be a choline salt, an L-lysine salt, a tert-butylamine salt, a diethanolamine salt, or a meglumine salt of the compound of Formula (I).

In some embodiments, the pharmaceutically acceptable salt of the compound of Formula (I) may be a crystalline form of the L-lysine salt of the compound of Formula (I). This form may exhibit an X-ray powder diffraction pattern having at least three characteristic peaks, wherein the three characteristic peaks are selected from the group consisting of peaks at 4.23, 7.92, 10.59, 14.44, 15.25, 15.50, 19.32, 19.87, 21.30, 23.41, 23.94, 24.42, 25.30, 25.81, 26.00 degrees 2θ (±0.2 degrees 2θ).

In some embodiments, the pharmaceutically acceptable salt of the compound of Formula (I) may be a crystalline form of the L-lysine salt of the compound of Formula (I). This form may exhibit an X-ray powder diffraction pattern having at least four characteristic peaks, wherein the four characteristic peaks are selected from the group consisting of peaks at 4.23, 7.92, 10.59, 14.44, 15.25, 15.50, 19.32, 19.87, 21.30, 23.41, 23.94, 24.42, 25.30, 25.81, 26.00 degrees 2θ (±0.2 degrees 2θ).

In some embodiments, the pharmaceutically acceptable salt of the compound of Formula (I) may be a crystalline form of the L-lysine salt of the compound of Formula (I). This form may exhibit an X-ray powder diffraction pattern having at least five characteristic peaks, wherein the five characteristic peaks are selected from the group consisting of peaks at 4.23, 7.92, 10.59, 14.44, 15.25, 15.50, 19.32, 19.87, 21.30, 23.41, 23.94, 24.42, 25.30, 25.81, 26.00 degrees 2θ (±0.2 degrees 2θ).

In some embodiments, the crystalline form of the L-lysine salt of the compound of Formula (I) may exhibit an X-ray powder diffraction pattern as depicted in FIG. 1.

In some embodiments of this aspect of the disclosure, the crystalline form of the L-lysine salt of the compound of Formula (I) exhibits a melting point of about 197-203° C.

In some embodiments of this aspect of the disclosure, the pharmaceutically acceptable salt of the compound of Formula (I) may be a crystalline form of the tert-butylamine salt of the compound of Formula (I). This form may exhibit an X-ray powder diffraction pattern having at least three characteristic peaks, wherein the three characteristic peaks are selected from the group consisting of peaks at 13.59, 13.75, 17.93, 18.36, 18.44, 19.39, 20.26, 20.77, 21.22, 21.78, 21.84, 22.82, 23.34, 23.91, 26.12 degrees 2θ (±0.2 degrees 2θ).

In some embodiments of the first aspect of the disclosure, the pharmaceutically acceptable salt of the compound of Formula (I) may be a crystalline form of the tert-butylamine salt of the compound of Formula (I). This form may exhibit an X-ray powder diffraction pattern having at least four characteristic peaks, wherein the four characteristic peaks are selected from the group consisting of peaks at 13.59, 13.75, 17.93, 18.36, 18.44, 19.39, 20.26, 20.77, 21.22, 21.78, 21.84, 22.82, 23.34, 23.91, 26.12 degrees 2θ (±0.2 degrees 2θ).

In some embodiments of the first aspect of the disclosure, the pharmaceutically acceptable salt of the compound of Formula (I) may be a crystalline form of the tert-butylamine salt of the compound of Formula (I). This form may exhibit an X-ray powder diffraction pattern having at least five characteristic peaks, wherein the five characteristic peaks are selected from the group consisting of peaks at 13.59, 13.75, 17.93, 18.36, 18.44, 19.39, 20.26, 20.77, 21.22, 21.78, 21.84, 22.82, 23.34, 23.91, 26.12 degrees 2θ (±0.2 degrees 2θ).

In some embodiments, the crystalline form of the tert-butylamine salt of the compound of Formula (I) may exhibit an X-ray powder diffraction pattern as depicted in FIG. 3.

In some embodiments, the crystalline form of the tert-butylamine salt of the compound of Formula (I) exhibits a melting point of about 196-224° C.

In some embodiments, any one of the pharmaceutically acceptable salts of the compound of Formula (I) and crystalline forms thereof listed above and a pharmaceutically acceptable excipient.

In some embodiments, the pharmaceutically acceptable salt of the compound of Formula (I) is the L-lysine salt of the compound of Formula (I). In other embodiments, the pharmaceutically acceptable salt of the compound of Formula (I) is the tert-butylamine salt of the compound of Formula (I).

In some embodiments of this aspect of the disclosure, disclosed here is a method of treating an eye disease comprising administering to a subject a therapeutically effective amounts of the pharmaceutically acceptable salts of the compound of Formula (I) and crystalline forms thereof.

In some embodiments, disclosed herein a method of treating an eye disease comprising administering to a subject a therapeutically effective amount of a pharmaceutically acceptable salt of the compound of Formula (I) and crystalline forms thereof disclosed herein, wherein the eye disease may be diabetic retinopathy, proliferative vitreoretinopathy corneal edema, anterior and posterior uveitis, pterygium, corneal diseases, dry eye, conjunctivitis, allergy- and laser-induced exudation, non-age related macular degeneration, macular edema, age-related macular degeneration, or ocular von Hippel-Lindau disease.

In some embodiments, disclosed herein is a method of reducing at least one of: the level of ICAM-1 mRNA in the retina of the subject; the level of VEGF mRNA in the retina of the subject; and the number of leukocytes in the retina of the subject, the method of administering to a subject a therapeutically effective amount of any one of the salts of the compound of Formula (I) and crystalline forms thereof disclosed herein, or the pharmaceutical composition comprising the pharmaceutically acceptable salts of the compound of Formula (I) and crystalline forms thereof disclosed herein.

In some embodiments, disclosed herein is a method of treating diseases or conditions associated with fibrosis, including but not limited to diseases selected from fibrotic skin disorders, such as keloids, hypertrophic scars and scleroderma; lung disease, such as pulmonary fibrosis; heart disease, such as heart failure due to ischaemic heart disease, valvular heart disease and hypertensive heart disease, diabetic cardiomyopathy and hypertension; and kidney disease, such as progressive kidney disease, due to, glomerulonephritis and diabetic nephropathy and cirrhosis of the liver, said method comprising administering to a subject a therapeutically effective amount of a pharmaceutically acceptable salt of the compound of Formula (I) and crystalline forms thereof disclosed herein. In some embodiments, the disease or condition is diabetic heart disease or diabetic kidney disease. In some embodiments, the disease or condition is diabetic cardiomyopathy. In some embodiments, the kidney disease may include a progressive glomerular kidney disease including without limitation diabetic nephropathy (e.g., as a consequence of Type I or Type II diabetes or systemic lupus), primary glomerulonephritis (e.g., membranous nephropathy, focal segmental glomerulosclerosis, membranoproliferative glomerulonephritis, diffuse proliferative glomerulonephritis, membranous focal segmental glomerulosclerosis) or secondary glomerulonephritis (e.g., diabetic nephropathy, ischemic nephropathy). In some embo

In some embodiments of the second aspect of the disclosure, any one of the methods listed above, wherein the pharmaceutically acceptable salts of the compound of Formula (I) and crystalline forms thereof disclosed herein is administered orally.

In some embodiments of the second aspect of the disclosure, any one of the methods listed above, wherein the pharmaceutically acceptable salts of the compound of Formula (I) and crystalline forms thereof disclosed herein, or compositions comprising the same, is administered to the eye.

In some embodiments, a method of treating an eye disease comprising administering to a subject a therapeutically effective amount of any one of the L-lysine salts of the compound of Formula (I) and crystalline forms thereof and/or the tert-butylamine salts of the compound of Formula (I) and crystalline forms thereof; or combinations thereof.

In some embodiments of the second aspect of the disclosure, a method of treating an eye disease comprising administering to a subject a therapeutically effective amount of any one of the L-lysine salts of the compound of Formula (I) and polymorphs thereof and the tert-butylamine salts of the compound of Formula (I) and polymorphs thereof, wherein the eye disease may be diabetic retinopathy, proliferative vitreoretinopathy corneal edema, anterior and posterior uveitis, pterygium, corneal diseases, dry eye, conjunctivitis, allergy- and laser-induced exudation, non-age related macular degeneration, macular edema, age-related macular degeneration or ocular von Hippel-Lindau disease.

In some embodiments, disclosed herein is a method of reducing at least one of: the level of ICAM-1 mRNA in the retina of the subject; the level of VEGF mRNA in the retina of the subject; and the number of leukocytes in the retina of the subject, the method comprising administering to a subject a therapeutically effective amount of any one of the L-lysine salts of the compound of Formula (I) and crystalline forms thereof; or a therapeutically effective amount of any one of the tert-butylamine salts of the compound of Formula (I) and crystalline forms thereof.

In some embodiments, disclosed herein is a method of treating diseases or conditions associated with fibrosis comprising administering to a subject a therapeutically effective amount of any one of the L-lysine salts of the compound of Formula (I) and crystalline forms thereof; or a therapeutically effective amount of any one of the tert-butylamine salts of the compound of Formula (I) and crystalline forms thereof. In some embodiments the fibrotic disease or disorder may be, for example, a fibrotic skin disorder selected from keloids, hypertrophic scars and scleroderma. In some embodiments, the disease or disorder associated with fibrosis may be pulmonary fibrosis. In some embodiments, the disease or disorder associated with fibrosis may be a heart disease. In some embodiments, the heart disease may be heart failure due to ischaemic heart disease, valvular heart disease and hypertensive heart disease, diabetic cardiomyopathy and hypertension. In some embodiments, the disease or disorder associated with fibrosis may be a heart disease. In some embodiments, the kidney disease may be progressive kidney disease due to glomerulonephritis and diabetic nephropathy and cirrhosis of the liver. In some embodiments, the kidney disease may be progressive glomerular kidney disease, diabetic nephropathy, primary glomerulonephritis or secondary glomerulonephritis. In some embodiments, the primary glomerulonephritis may be membranous nephropathy, focal segmental glomerulosclerosis, membranoproliferative glomerulonephritis, diffuse proliferative glomerulonephritis, or membranous focal segmental glomerulosclerosis. In some embodiments, the secondary glomerulonephritis may be diabetic nephropathy or ischemic nephropathy. In some embodiments, the kidney disease may include progressive kidney diseases with origins primarily in the tubulointerstitium. In some specific embodiments, the kidney disease may be chronic interstitial nephritis, autosomal dominant tubulointerstitial fibrosis, or reflux nephropathy.

In some embodiments the pharmaceutically acceptable salts of the compound of Formula (I) and crystalline forms thereof may be administered orally.

In other embodiments, pharmaceutically acceptable salts of the compound of Formula (I) and crystalline forms thereof may be administered to the eye.

### BRIEF DESCRIPTION OF THE DRAWINGS

FIG. 1 depicts an example of an X-ray powder diffraction pattern of a crystalline form of a L-lysine salt of the compound of Formula (I).

FIG. 2 depicts an example of a differential thermal analysis and thermogravimetric analysis overlay for a crystalline form of a L-lysine salt of the compound of Formula (I).

FIG. 3 depicts an example of an X-ray powder diffraction pattern of crystalline Form I of a tert-butylamine salt of the compound of Formula (I).

FIG. 4 depicts an example of a differential thermal analysis and thermogravimetric analysis overlay for crystalline Form I of a tert-butylamine salt of the compound of Formula (I).

FIG. 5 depicts an example of a dynamic vapor sorption result for a crystalline form of a L-lysine salt of the compound of Formula (I).

FIG. 6 depicts an example of a dynamic vapor sorption result for a crystalline form of a tert-butylamine salt of the compound of Formula (I).

FIG. 7 depicts an example of an X-ray powder diffraction pattern of crystalline Form II of a tert-butylamine salt of the compound of Formula (I).

FIG. 8 depicts an example of an X-ray powder diffraction pattern of crystalline Form III of a tert-butylamine salt of the compound of Formula (I).

FIG. 9 depicts an example of a differential scanning calorimetry (DSC) data for crystalline Form III of a tert-butylamine salt of the compound of Formula (I).

FIG. 10 depicts an example of an X-ray powder diffraction pattern of crystalline Form IV of a tert-butylamine salt of the compound of Formula (I).

FIG. 11 depicts an example of a differential scanning calorimetry (DSC) data for crystalline Form IV of a tert-butylamine salt of the compound of Formula (I).

FIG. 12 depicts an example of a dissolution profile of a crystalline form of a L-lysine salt of the compound of Formula (I) and a crystalline form of a tert-butylamine salt of the compound of Formula (I) in Fasted Stated Simulated Intestinal Fluid (FaSSIF) media.

FIG. 13 depicts an example of a dissolution profile of a crystalline form of a L-lysine salt of the compound of Formula (I) and a crystalline form of a tert-butylamine salt of the compound of Formula (I) in Phosphate-buffered saline (PBS) media.

### DETAILED DESCRIPTION OF THE PREFERRED EMBODIMENT

### Definitions

In this specification a number of terms are used which are well known to a person of skill in the art. Nevertheless, for the purposes of clarity a number of terms will be defined.

The term “pharmaceutically acceptable salts,” as used herein, refers to salts that retain the desired biological activity of the compound of Formula (I). Suitable pharmaceutically acceptable salts of the compound of Formula (I) and polymorphs thereof include metallic salts made from lithium, sodium, potassium, magnesium, calcium, aluminium, and zinc, and organic salts made from organic bases such as choline, diethanolamine, or morpholine. Other examples of organic salts are: ammonium salts such as tert-butylamine and diethanolamine; quaternary salts such as tetramethylammonium salt and choline salt; amino acid addition salts such as salts with glycine, arginine, and L-lysine. Additional information on pharmaceutically acceptable salts can be found in Remington's Pharmaceutical Sciences, 19th Edition, Mack Publishing Co., Easton, Pa. 1995. In the case of agents that are solids, it is understood by those skilled in the art that the disclosed compound of Formula (I), agents and salts may exist in different crystalline or polymorphic forms, all of which are intended to be within the scope of the present disclosure and specified formula.

The terms “treating,” “treat,” or “treatment,” as used herein, refer generally to amelioration or elimination of a named condition once the condition has been established. The term “prophylaxis,” as used herein, refers generally to treatment to prevent the onset of a named condition or of a process that can lead to the condition (“primary” prophylaxis), or the recurrence of symptoms of a condition.

The term “subject,” as used herein, refers generally to any warm-blooded animal such as, but not limited to, a mouse, guinea pig, dog, horse, or human. In a preferred embodiment, the subject is a human.

The term “therapeutically effective amount” or “effective amount,” as used herein, is an amount sufficient to effect beneficial or desired clinical results. An effective amount can be administered in one or more administrations. An effective amount is typically sufficient to palliate, ameliorate, stabilize, reverse, slow or delay the progression of the disease state.

The term “pharmaceutically acceptable,” as used herein, refers generally to a substance or composition that is compatible chemically and/or toxicologically with the other ingredients including a formulation, and/or the subject being treated.

The term “suitable solvent,” as used herein, refers generally to a single solvent as well as mixtures of solvents. Solvents may be selected, as appropriate for a given reaction step, from, for example, aprotic polar solvents such as DMF, DMA, DMSO, dimethylpropyleneurea, N-methylpyrrolidone (NMP), and hexamethylphosphoric triamide; ether solvents such as diethyl ether, THF, 1,4-dioxane, methyl t-butyl ether, dimethoxymethane, and ethylene glycol dimethyl ether; alcohol solvents such as MeOH, EtOH, and isopropanol; and halogen-containing solvents such as methylene chloride, chloroform, carbon tetrachloride, and 1,2-dichloroethane. Mixtures of solvents may also include biphasic mixtures.

### General Overview

Disclosed herein are pharmaceutically acceptable salts of the compound of Formula (I) and crystalline forms thereof. The compound of Formula (I) is show below:

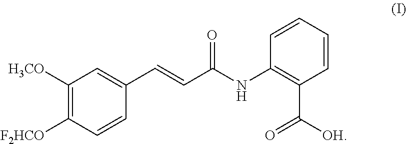 [SMILES: COc1cc(/C=C/C(=O)Nc2ccccc2C(=O)O)ccc1OC(F)F]

Some embodiments include the salts of the compound of Formula (I) and crystalline forms thereof, wherein the acceptable salt may be, but not limited to, a sodium salt, a potassium salt, a zinc salt, a magnesium salt, a calcium salt, a choline salt, a L-lysine salt, a tert-butylamine salt, and a diethanolamine salt.

Other salt forms of Formula (I) include L-lysine salts and crystalline forms thereof and tert-butylamine salts and crystalline forms thereof described herein.

The present application also relates to the method using the various salts of the compound of Formula (I) and polymorphs thereof, particularly for treating diseases and disorders by administering to a patient a therapeutically effective amount of a composition comprising one or more salt forms of the compound of Formula (I), and one or more pharmaceutically acceptable excipients.

The present application also relates to methods of crystallizing the various salt forms of the compound of Formula (I) and crystal forms thereof. The salt forms advantageously exhibit improved solubility, bioavailability, stability, processability and ease of manufacture. As a result, the salt forms of the compound of Formula (I) and crystalline forms thereof, provide long-term stability, low adsorption and desorption of water vapor, and superior dissolution properties compared to the free acid form of the compound of Formula (I).

### Salts and Crystalline Forms of the Salts of the Compound of Formula (I)

Disclosed herein are salts of the compound of Formula (I) and crystalline forms thereof.

Some embodiments include the salts of the compound of Formula (I) and polymorphs thereof, wherein the acceptable salt may be, but not limited to, a sodium salt, a potassium salt, a zinc salt, a magnesium salt, or a calcium salt.

Some embodiments include the sodium salt of the compound of Formula (I) and crystalline forms thereof.

Some embodiments include the potassium salt of the compound of Formula (I) and crystalline forms thereof.

Some embodiments include the zinc salt of the compound of Formula (I) and crystalline forms thereof.

Some embodiments include the magnesium salt of the compound of Formula (I) and crystalline forms thereof.

Some embodiments include the calcium salt of the compound of Formula (I) and crystalline forms thereof.

Some embodiments include the salt of the compound of Formula (I), wherein the acceptable salt may be, but not limited to, a choline salt, an L-lysine salt, a tert-butylamine salt, or a diethanolamine salt.

Some embodiments include the choline salt of the compound of Formula (I) and crystalline forms thereof.

Some embodiments include the L-lysine salt of the compound of Formula (I) and crystalline forms thereof.

Some embodiments include the tert-butylamine salt of the compound of Formula (I) and crystalline forms thereof.

Some embodiments include the diethanolamine salt of the compound of Formula (I) and crystalline forms thereof.

### Crystalline Forms of the Compound of Formula (I)

Disclosed herein are salts of the compound of Formula (I) and crystalline forms thereof. Unless otherwise stated, the X-ray powder diffraction data provided herein was determined using a Cu Kα radiation source.

In some embodiments, the crystalline forms of the salts of the compound of Formula (I) may be solvated. In some embodiments, the crystalline forms of the salts of the compound of Formula (I) may be a hydrate. In some embodiments, the crystalline forms of the salts of the compound of Formula (I) may be unsolvated.

### Crystalline Form of the L-Lysine Salt of the Compound of Formula (I) (“Crystalline Salt A”)

Some embodiments include an unsolvated L-lysine salt form of the compound of Formula (I), which is optionally referred to herein as Crystalline Salt A. The precise conditions for forming Crystalline Salt A may be empirically determined, but exemplary methods disclosed herein have been found to be suitable, in practice, to form Crystalline Salt A.

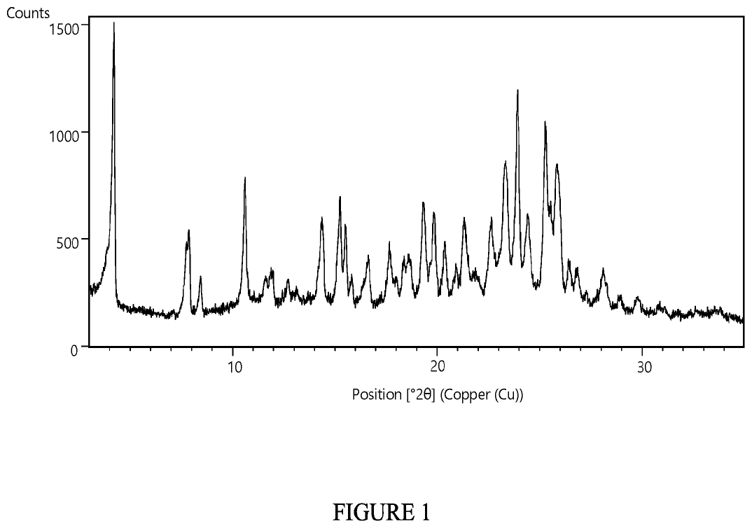

Crystalline Salt A is characterized, herein, using various techniques which are described in further detail in the experimental methods section. FIG. 1 depicts an example of an X-ray powder diffraction (XRPD) pattern for Crystalline Salt A. As shown in FIG. 1, Crystalline Salt A, which may be obtained by the methods disclosed herein, exhibits prominent peaks at approximately 4.23, 7.92, 10.59, 14.44, 15.25, 15.50, 19.32, 19.87, 21.30, 23.41, 23.94, 24.42, 25.30, 25.81, 26.00 degrees 2θ. Thus, in some embodiments, a crystalline form of the compounds of Formula (I) has at least one characteristic peak (e.g., one, two, three, four, five, six, seven, eight, nine, ten or eleven characteristic peaks) selected from approximately 4.23, 7.92, 10.59, 14.44, 15.25, 15.50, 19.32, 19.87, 21.30, 23.41, 23.94, 24.42, 25.30, 25.81, 26.00 degrees 2θ. In some embodiments, a crystalline form of the compounds of Formula (I) has at least three characteristic peaks selected from approximately 4.23, 7.92, 10.59, 14.44, 15.25, 15.50, 19.32, 19.87, 21.30, 23.41, 23.94, 24.42, 25.30, 25.81, 26.00 degrees 2θ.

As will be understood by persons of skill in the art, because of the experimental variability when X-ray diffraction patterns are measured on different instruments, the peak positions are assumed to be equal if the two theta (20) values agree to within a certain degree of variability. For example, the United States Pharmacopeia states that if the angular setting of the 10 strongest diffraction peaks agree to within ±0.2 degrees with that of a reference material, and the relative intensities of the peaks do not vary by more than 20%, the identity is confirmed. Accordingly, in some embodiments, peak positions recited herein include variability within ±0.5 degrees 2θ. In other embodiments, peak positions recited herein include variability within ±0.2 degrees 2θ. As disclosed herein, the term “approximately” when referring to values of 20 is defined as ±0.5 degrees 2θ.

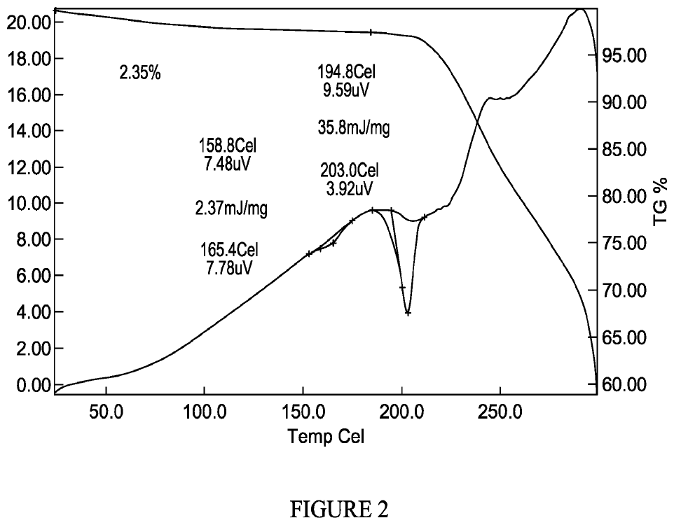

FIG. 2 depicts an example of a differential thermal analysis (DTA) and thermogravimetric analysis (TG) overlay for Crystalline Salt A. In one instance, Crystalline Salt A exhibited a 2.3% weight loss when carried out from 25° C. to 300° C. The DTA analysis indicate a melting range of about 197-203° C. for Crystalline Salt A.

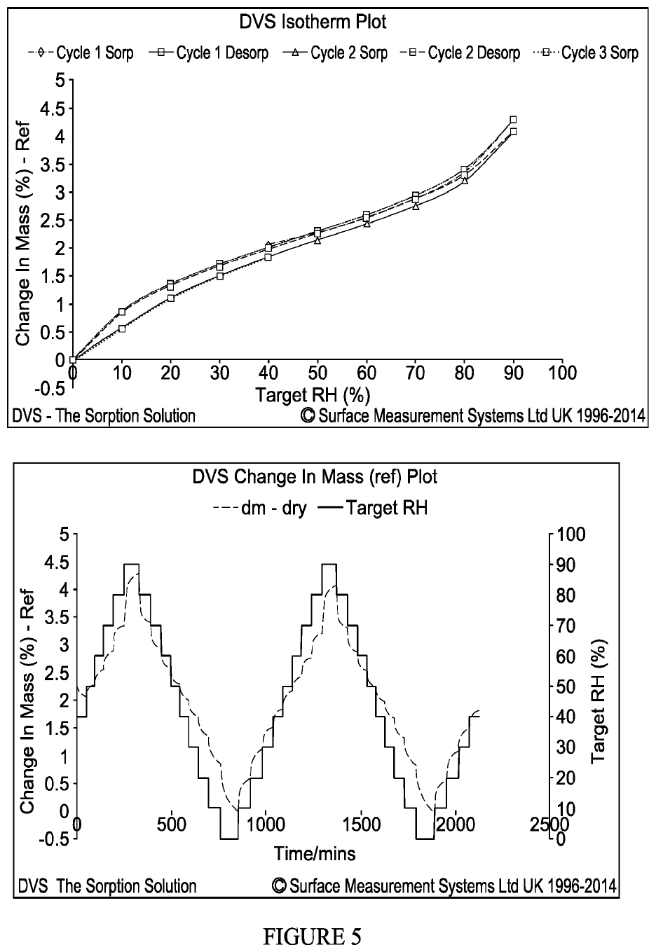

FIG. 5 depicts an example of a dynamic vapor sorption (DVS) analysis for Crystalline Salt A, and shows a mass increase of 4.2 wt. % at 90% Relative Humidity (RH) (1.3 equiv. of water).

Crystalline Salt A may therefore be characterized as stable over a wide range of humidity. Crystalline Salt A also shows good crystallinity, the melting point is relatively high (approx. 200° C.) and Crystalline Salt A does not show any evidence of hydrate formation.

### Crystalline Form of the Tert-Butylamine Salt of the Compound of Formula (I) (“Crystalline Salt B”)

Some embodiments include an unsolvated tert-butylamine salt of the compound of Formula (I), referred to herein as Crystalline Salt B. The precise conditions for forming Crystalline Salt B may be empirically determined and it is only possible to give a number of methods which have been found to be suitable in practice.

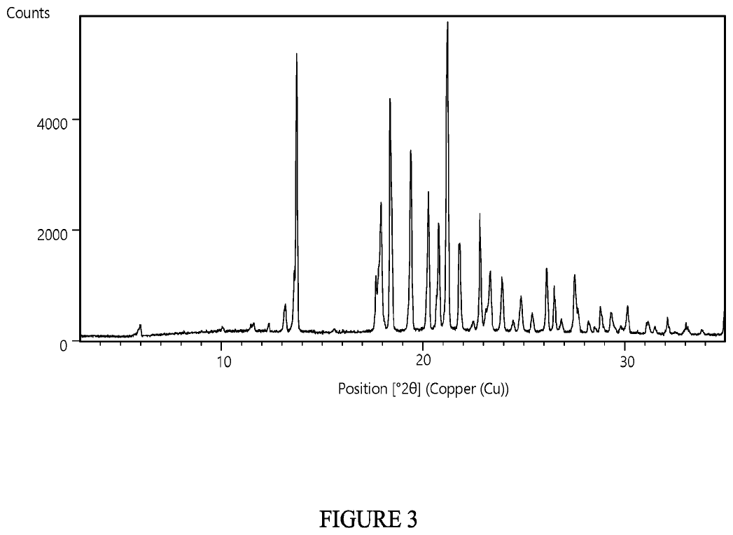

Crystalline Salt B was characterized using various techniques which are described in further detail in the experimental methods section. FIG. 3 depicts an example of an X-ray powder diffraction (XRPD) pattern for the salt structure of Form I of Crystalline Salt B. Crystalline Salt B, which may be obtained by the methods disclosed herein, exhibits prominent peaks at approximately 13.59, 13.75, 17.93, 18.36, 18.44, 19.39, 20.26, 20.77, 21.22, 21.78, 21.84, 22.82, 23.34, 23.91, 26.12 degrees 2θ. Thus, in some embodiments, a crystalline form of the compounds of Formula (I) has at least one characteristic peak (e.g., one, two, three, four, five, six, seven, eight, nine, ten or eleven characteristic peaks) selected from approximately 13.59, 13.75, 17.93, 18.36, 18.44, 19.39, 20.26, 20.77, 21.22, 21.78, 21.84, 22.82, 23.34, 23.91, 26.12 degrees 2θ. In some embodiments, a crystalline form of the compounds of Formula (I) has at least three characteristic peaks selected from approximately 13.59, 13.75, 17.93, 18.36, 18.44, 19.39, 20.26, 20.77, 21.22, 21.78, 21.84, 22.82, 23.34, 23.91, 26.12 degrees 2θ.

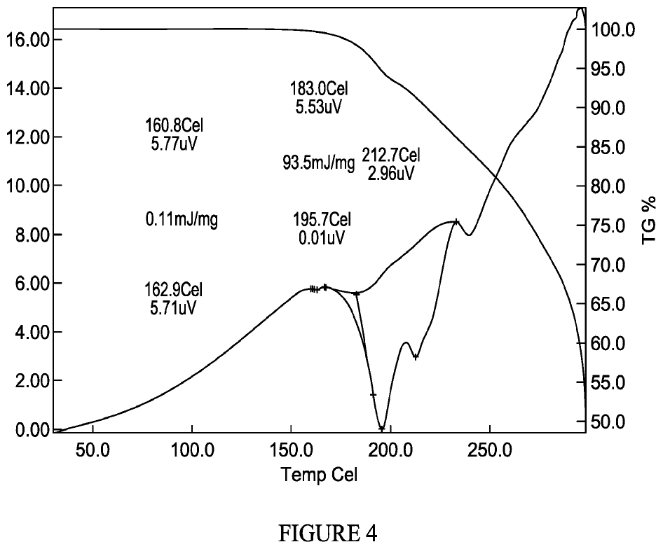

FIG. 4 depicts an example of a differential thermal analysis (DTA) and thermogravimetric analysis (TG) overlay for Form I of Crystalline Salt B. In one instance, Crystalline Salt B exhibited no significant mass loss until after the onset of thermal degradation. The DTA analysis indicates a melting point range of about 196-224° C. for Crystalline Salt B.

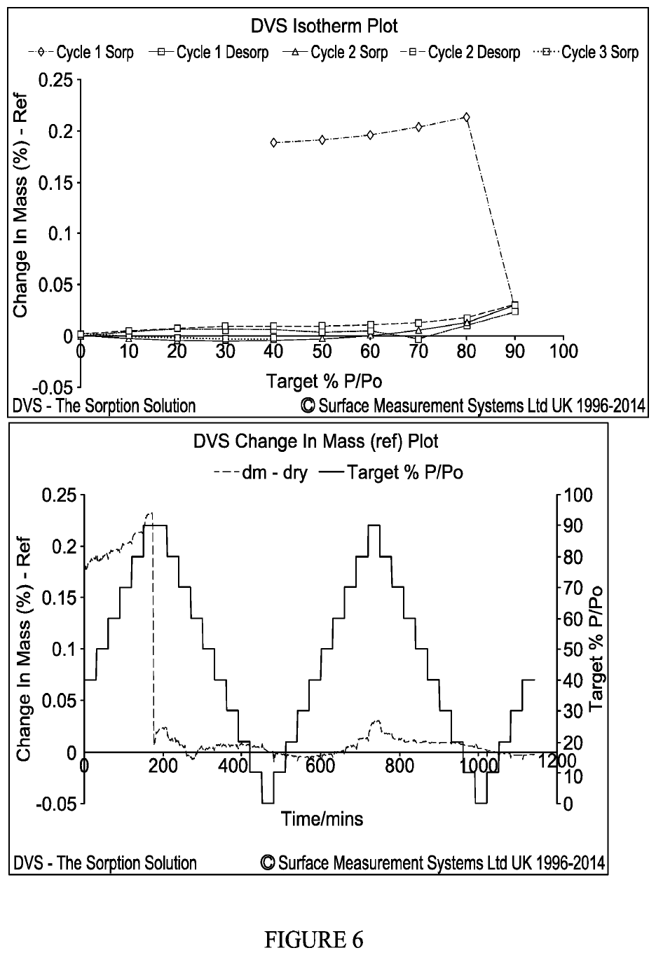

FIG. 6 depicts an example of a dynamic vapor sorption (DVS) analysis for Form I of Crystalline Salt B, and shows negligible water uptake at 90% RH.

Form I of Crystalline Salt B may therefore be characterized as non-hygroscopic and stable over a wide range of humidity. Crystalline Salt B also shows good crystallinity, the melting point is relatively high (approx. 210° C.) and Crystalline Salt B does not show any evidence of hydrate formation.

### Methods of Making the Crystalline Forms of Formula (I)

Salt forms of the compound of Formula (I), including for example the L-Lysine salt form, may be prepared by the procedure outlined below. The compound of Formula (I) is dissolved in a minimum amount of solvent selected from 2-propanol, acetonitrile, ethanol, ethyl acetate, methanol and isopropyl acetate. L-lysine (1.05 equivalents) is then added and the sample temperature cycled between ambient temperature and an elevated temperature, for example, 40° C., in fixed-time cycles, for example, 4-hour cycles for an extended duration, for example 72 hours. After such a time, an anti-solvent, selected from the list of heptane or toluene, is added to the mixture and the samples stored at 2-8° C. to promote precipitation. The L-lysine salt of the compound of Formula (I) may be isolated as crystalline material via centrifugation filtration, in particular from the solvent mixture consisting of methanol/toluene. If no crystalline material is obtained the solvents are allowed to evaporate under ambient conditions. At this time, a salt form, for example the L-Lysine salt form, of the compound of Formula (I) may be obtained as crystalline material, in particular from solvent mixtures consisting of 2-propanol/heptane, acetonitrile/toluene, and ethanol/heptane. The observed solids were dried under vacuum at elevated temperature, for example, 40° C., for an extended period, for example, 6 hours.

Alternative salt forms of the compound of Formula (I), including for example the tert-butylamine salt of the compound of Formula (I), may be prepared by the procedure outlined below. The compound of Formula (I) is dissolved in a minimum amount of solvent selected from 2-propanol, acetonitrile, ethanol, ethyl acetate, methanol and isopropyl acetate. tert-Butylamine is then added and the sample temperature cycled between ambient temperature at elevated temperature, for example, 40° C., in fixed-time cycle, for example 4-hour cycles, for an extended duration, for example 72 hours. After such time, a salt form, for example the tert-butylamine salt form, of the compound of Formula (I) may be obtained as crystalline material by filtration, particularly if using a solvent selected from the list 2-propanol, acetonitrile, ethyl acetate and isopropyl acetate. If no crystalline material is obtained, an anti-solvent, selected from the list of heptane or toluene, is added to the mixture and the solvent allowed to evaporate under ambient conditions. After this time the tert-butylamine salt of the compound of Formula (I) may be obtained as crystalline material. The observed solids are dried under vacuum at elevated temperature, for example, 40° C. for an extended duration, for example 6 hours. Other crystalline forms of tert-butylamine salt of the compound of Formula (I), such as Form II, Form III, and Form IV, may be prepared by using 1,1,1,3,3,3-hexafluoroisopropanol (HFIPA) as a solvent.

Non-limiting examples of preparing the various crystalline forms of the salts of the compound of Formula (I) are provided in the Examples.

### Formulations of Pharmaceutical Compositions

The salts of the compound of Formula (I) and crystalline forms thereof (for example, Crystalline Salt A, Crystalline Salt B, as disclosed herein) can be provided as combinations with other therapeutic agents or in pharmaceutical compositions. The pharmaceutical compositions provided herein include therapeutically effective amounts of salt forms of the compound of Formula (I) and crystalline forms thereof.

The compositions provided herein may include one or more salt forms of the compound of Formula (I) and crystalline forms thereof provided herein, alone or in combination or in admixture. The compositions are formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically, the compounds described above are formulated into pharmaceutical compositions using techniques and procedures well known in the art (see, e.g., Ansel, Introduction to Pharmaceutical Dosage Forms, 4^{th} Edition (1985), 126).

In some embodiments, the composition comprising the salt of the compound of Formula (I) and crystalline forms thereof may include one or more salts of the compound of Formula (I) and crystalline forms thereof. In some embodiments, the total amount of the salt of the compound of Formula (I) and crystalline forms thereof in the composition may include at least about 50% by weight of a salt form of the compound of Formula (I). In some embodiments, the total amount of salt of the compound of Formula (I) and crystalline forms thereof in the composition may include at least about 80% by weight of a salt form of the compound of Formula (I). In some embodiments, the total amount of salt of the compound of Formula (I) and crystalline forms in the composition may include at least about 95%, 96%, 97%, 98%, or 99% by weight of a salt form of the compound of Formula (I).

In some embodiments, the total amount of the salt of the compound of Formula (I) in the composition may include at least about 50% by weight of Crystalline Salt A. In some embodiments, the total amount of the salt of the compound of Formula (I) in the composition may include at least about 80% by weight of Crystalline Salt A. In some embodiments, the total amount of the salt of the compound of Formula (I) in the composition may include at least about 95% by weight of Crystalline Salt A. In some embodiments, the total amount of the salt of the compound of Formula (I) in the composition may include at least about 96%, 97%, 98%, or 99% by weight of Crystalline Salt A.

In some embodiments, the total amount of the salt of the compound of Formula (I) in the composition may include at least about 50% by weight of Crystalline Salt B. In some embodiments, the total amount of the salt of the compound of Formula (I) in the composition may include at least about 80% by weight of Crystalline Salt B. In some embodiments, the total amount of the salt of the compound of Formula (I) in the composition may include at least about 95% by weight of Crystalline Salt B. In some embodiments, the total amount of the salt of the compound of Formula (I) in the composition may include at least about 96%, 97%, 98%, or 99% by weight of Crystalline Salt B.

The compositions, in some embodiments, include Crystalline Salt A. For example, the total amount of the salt of the compound of Formula (I) and polymorphs thereof in the composition may include at least about 20%; at least about 50%; at least about 90%; at least about 95%; or at least about 99% of Crystalline Salt A. Similarly, the compositions may also include, for example, Crystalline Salt B. The amount of the total amount of the salt of the compound of Formula (I) and polymorphs thereof in the composition may include at least about 20%; at least about 50%; at least about 90%; at least about 95%; or at least about 96%, 97%, 98%, or 99% of Crystalline Salt B.

In certain embodiments, a pharmaceutical composition including one or more salts and polymorphs thereof provided herein is prepared using known techniques, including, but not limited to, mixing, dissolving, granulating, dragée-making, levigating, emulsifying, encapsulating, entrapping or tableting processes.

In the compositions, effective concentrations of one or more salt of the compound of Formula (I) and crystalline forms thereof described herein is (are) mixed with a suitable pharmaceutical carrier or vehicle. The concentrations of the compounds in the compositions are effective for delivery of an amount, upon administration, that treats, prevents, or ameliorates one or more of the symptoms of fibrotic diseases or disorders, including those described herein.

Typically, the compositions are formulated for single dosage administration. To formulate a composition, the weight fraction of the salts and crystalline forms thereof is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the salts and polymorphs thereof can be formulated as the sole pharmaceutically active ingredient in the composition or can be combined with other active ingredients. Liposomal suspensions, including tissue-targeted liposomes, such as tumor-targeted liposomes, also can be suitable as pharmaceutically acceptable carriers. These can be prepared according to methods known to those skilled in the art. For example, liposome formulations can be prepared as described in U.S. Pat. No. 4,522,811. Briefly, liposomes such as multilamellar vesicles (MLV's) can be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

One or more than one of the salts and crystalline forms thereof provided herein is included in the pharmaceutically acceptable carrier in an amount sufficient to exert a therapeutically useful effect in the absence of undesirable side effects on the subject treated.

The concentration of the one or more than one salt and crystalline forms thereof provided herein in the pharmaceutical composition will depend on absorption, inactivation and excretion rates of the compound, the physicochemical characteristics of the compound, the dosage schedule, and amount administered as well as other factors known to those of skill in the art. For example, the amount that is delivered is sufficient to ameliorate one or more of the symptoms of fibrotic diseases or disorders, including those described herein.

The effective amount of a salt of or crystalline form thereof provided herein can be determined by one of ordinary skill in the art, and includes exemplary dosage amounts for a mammal of from about 0.05 to 100 mg/kg of body weight of active compound per day, which can be administered in a single dose or in the form of individual divided doses, such as from 1 to 4 times per day. It will be understood that the specific dose level and frequency of dosage for any particular subject can be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. In some embodiments, the daily dosage of a salt of the compound of Formula (I) and crystalline forms thereof provided herein can be varied over a wide range from about or 0.01 to about or 1000 mg per adult human per day. For example, dosages can range from about or 0.1 to about or 200 mg/day. In some embodiments, the dosage can range from 0.2 mg to 20 mg per day. In some embodiments, the dosage can range from 0.5 mg to 10 mg per day. Small doses (0.01-1 mg/kg per day) may be administered initially, followed by increasing doses up to about 1000 mg/kg per day. In the event that the response in a subject is insufficient at such doses, even higher doses (or effective higher doses by a different, more localized delivery route) may be employed to the extent patient tolerance permits. A more preferred dosage will be in the range from 0.1 to 300 mg per kilogram of body weight per day, more preferably from 0.1 to 100 mg per kilogram of body weight per day. A suitable dose can be administered in multiple sub-doses per day. In some embodiments, the daily dosage can be 0.01 mg, 0.05 mg, 0.1 mg, 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.25 mg, 5.5 mg, 5.75 mg, 6 mg, 6.25 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg. For oral administration, the compositions can be provided in the form of unit dosages such as tablets or capsules or liquids including from about or 0.01 to about or 1000 mg, such as for example, 0.01, 0.05, 0.075, 0.1, 0.25, 0.5, 0.75, 1, 2, 2.5, 3, 4, 5, 6, 7, 8, 9, 10, 15, 20, 25, 50, 75, 100, 125, 150, 175, 180, 190, 200, 225, 250, 300, 400, 500, 750, 800, 850, 900, 950 and 1000 milligrams of the active ingredient for the symptomatic adjustment of the dosage to the subject to be treated. In some embodiments, the compositions can be provided in the form of unit dosages such as tablets or capsules or liquids including from about or 0.01 to about or 1000 μg, such as for example, 0.01, 0.05, 0.075, 0.1, 0.25, 0.5, 0.75, 1, 2.5, 3, 4, 5, 6, 7, 8, 9, 10, 15, 20, 25, 50, 75, 100, 125, 150, 175, 180, 190, 200, 225, 250, 300, 350, 400, 450, 500, 550, 600, 650, 700, 750, 800, 850, 900, 950 and 1000 micrograms of the active ingredient for the symptomatic adjustment of the dosage to the subject to be treated.

The pharmaceutical composition including one or more than one salt and crystalline form thereof provided herein can be administered at once, or can be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the salts and polymorphs thereof, compositions, methods and other subject matter provided herein.

Thus, effective concentrations or amounts of one or more of the salts and crystalline forms thereof described herein are mixed with a suitable pharmaceutical carrier or vehicle for systemic, topical or local administration to form pharmaceutical compositions. One or more than one salt and crystalline forms thereof provided herein is/are included in an amount effective for ameliorating one or more symptoms of, or for treating or preventing diseases or disorders described herein. The concentration of the one or more than one salt and crystalline forms thereof in the composition will depend on absorption, inactivation, excretion rates of the active compound, the dosage schedule, amount administered, particular formulation as well as other factors known to those of skill in the art.

The compositions are intended to be administered by a suitable route, including orally in the form of capsules, tablets, granules, powders or liquid formulations including syrups; parenterally, such as subcutaneously, intravenously, intramuscularly, with intersternal injection or infusion techniques (as sterile injectable aqueous (aq.) or non-aqueous solutions or suspensions); nasally, such as by inhalation spray; topically, such as in the form of a cream or ointment; rectally, such as in the form of suppositories; liposomally; and locally. The compositions can be in liquid, semi-liquid or solid form and are formulated in a manner suitable for each route of administration. In certain embodiments, administration of the formulation includes parenteral and oral modes of administration. In one embodiment, the compositions are administered orally. In another embodiment, the compositions are administered directly to the eye via injection.

In certain embodiments, the pharmaceutical compositions provided herein including one or more salt and crystalline forms there thereof provided herein is a solid (e.g., a powder, tablet, and/or capsule). In certain of such embodiments, a solid pharmaceutical composition including one or more salt and polymorphs thereof provided herein is prepared using ingredients known in the art, including, but not limited to, starches, sugars, diluents, granulating agents, gums, lubricants, binders, and disintegrating agents.

In certain embodiments, the pharmaceutical compositions provided herein including one or more salt and crystalline forms thereof provided herein are a liquid or semi-liquid. In certain of such embodiments, a liquid pharmaceutical composition including one or more salt form of the compound of Formula (I) may be prepared by dissolving the salt form of Formula (I) in a suitable solvent or mixtures of solvents including, but not limited to, water, methanol, ethanol, n-propanol, isopropanol, glycerol, propylene glycol, polyethylene glycol, acetone, dimethyl sulfoxide, chloroform, and isopropyl myristate. In other such embodiments, one or more salt forms of the compound of Formula (I) and crystalline forms thereof may be formulated as a suspension in a liquid dispersion medium comprising one or more liquids.

In certain embodiments, a pharmaceutical composition including one or more salt and crystalline forms thereof provided herein includes a delivery system. Examples of delivery systems include, but are not limited to, liposomes and emulsions. Certain delivery systems are useful for preparing certain pharmaceutical compositions including those including hydrophobic compounds. In certain embodiments, certain organic solvents such as dimethylsulfoxide are used.

In certain embodiments, a pharmaceutical composition including one or more salt and crystalline forms thereof provided herein (active ingredient) includes one or more tissue-specific delivery molecules designed to deliver the pharmaceutical composition to specific tissues or cell types. For example, in certain embodiments, pharmaceutical compositions include liposomes coated with a tissue-specific antibody.

In certain embodiments, a pharmaceutical composition including one or more salt and crystalline forms thereof provided herein includes a co-solvent system. Certain of such co-solvent systems include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™, and 65% w/v polyethylene glycol 300. The proportions of such co-solvent systems can be varied considerably without significantly altering their solubility and toxicity characteristics. Furthermore, the identity of co-solvent components can be varied: for example, other surfactants can be used instead of Polysorbate 80™; the fraction size of polyethylene glycol can be varied; other biocompatible polymers can replace polyethylene glycol, e.g., polyvinyl pyrrolidone; and other sugars or polysaccharides can substitute for dextrose.

In certain embodiments, solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediamine-tetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the salts and crystalline forms thereof exhibit insufficient solubility, methods for solubilizing compounds can be used. Such methods are known to those of skill in this art, and include, but are not limited to, using cosolvents, such as dimethylsulfoxide (DMSO), using surfactants, such as surfactants that include polyoxyethylene derivatives of sorbitan monolaurate, such as TWEEN® or polysorbate surfactants, or dissolution in aqueous sodium bicarbonate. Derivatives of the compounds, such as prodrugs of the compounds also can be used in formulating effective pharmaceutical compositions.

In certain embodiments, a pharmaceutical composition including one or more salt or crystalline form thereof provided herein includes a sustained release system. A non-limiting example of such a sustained-release system is a semipermeable matrix of solid hydrophobic polymers. In certain embodiments, sustained release systems can, depending on their chemical nature, release compounds over a period of hours, days, weeks or months.

In certain embodiments, upon mixing or addition of the salts and crystalline forms thereof described herein, the resulting mixture can be a solution, suspension or emulsion. The form of the resulting mixture depends upon a number of factors, including the intended mode of administration and the solubility of the salt described herein in the selected carrier or vehicle. The effective concentration is sufficient for ameliorating the symptoms of the disease, disorder or condition treated and can be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions including suitable quantities of the salts and polymorphs thereof or pharmaceutically acceptable derivatives thereof. The pharmaceutically active compounds and derivatives thereof are typically formulated and administered in unit dosage forms.

The composition can include, in addition to the one or more than one salt and crystalline forms thereof provided herein, other ingredients, such as, but not limited to, a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing the salts or crystalline forms thereof described herein and optional pharmaceutical adjuvants in a carrier, such as, for example, water, saline, aqueous dextrose, glycerol, glycols and ethanol, to thereby form a solution or suspension. If desired, the pharmaceutical composition to be administered also can include minor amounts of nontoxic auxiliary substances such as wetting agents, emulsifying agents, or solubilizing agents, or pH buffering agents, for example, acetate or sodium citrate, or cyclodextrin derivatives, sorbitan monolaurate, triethanolamine sodium acetate, triethanolamine oleate. Actual methods of preparing such dosage forms are known, or will be apparent, to those skilled in this art; for example, see Remington's Pharmaceutical Sciences, Mack Publishing Company, Easton, Pa., 15^{th} edition (1975). The composition or formulation to be administered will, in any event, include a quantity of the active compound in an amount sufficient to alleviate the symptoms of the treated subject.

Dosage forms or compositions can be prepared to include one or more than one salt and crystalline forms thereof provided herein in the range of 0.005% to 100% with the balance made up from non-toxic carrier. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium croscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions can include 0.001%-100% active ingredient, in one embodiment 0.1-85%, in another embodiment 75-95%. In some embodiments, the compositions include 1-10% active ingredient. In some embodiments, the compositions include 10-25% active ingredient. In some embodiments, the compositions include 15-35% active ingredient. In some embodiments, the compositions include 40-60% active ingredient. In some embodiments, the compositions include 50-75% active ingredient. In some embodiments, the active ingredient is present at 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In certain embodiments, the salts and crystalline forms thereof can be administered in a form suitable for immediate release or extended release. Immediate release or extended release can be achieved with suitable pharmaceutical compositions or, particularly in the case of extended release, with devices such as subcutaneous implants or osmotic pumps. Exemplary compositions for topical administration include a topical carrier such as a mineral oil gelled with polyethylene (e.g., PLASTIBASE®).

In certain embodiments, the pharmaceutical salts and crystalline forms thereof include one or more than one salt and polymorphs thereof provided herein in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

The compositions can include, in addition to the one or more than one salt and crystalline forms thereof provided herein, other active compounds to obtain desired combinations of properties. The salts and crystalline forms thereof provided herein also can be advantageously administered for therapeutic or prophylactic purposes together with another pharmacological agent known in the general art to be of value in treating one or more of the diseases, disorders, or medical conditions referred to hereinabove, such as eye diseases associated with, but not limited to, inflammation and/or vascular proliferation as described herein or fibrotic diseases or disorders, including those described herein. It is to be understood that such combination therapy constitutes a further aspect of the compositions and methods of treatment provided herein.

In certain embodiments, a pharmaceutical composition including one or more salt and crystalline forms thereof provided herein is useful for treating a conditions or disorder in a mammalian, and particularly in a human subject. Suitable administration routes include, but are not limited to, oral, rectal, transmucosal, intestinal, enteral, topical, suppository, through inhalation, intrathecal, intraventricular, intraperitoneal, intranasal, intraocular and parenteral (e.g., intravenous, intramuscular, intramedullary, and subcutaneous). In certain embodiments, pharmaceutical compositions are administered to achieve local rather than systemic exposures. For example, pharmaceutical compositions can be injected directly in the area of desired effect (e.g., in the eye or corneal area). In certain embodiments in which the pharmaceutical composition is administered locally, the dosage regimen is adjusted to achieve a desired local concentration of a salt or crystalline form thereof provided herein.

In certain embodiments, a pharmaceutical composition including one or more salts and crystalline forms thereof provided herein is administered in the form of a dosage unit (e.g., tablet, capsule, pill, injection, bolus).

In certain embodiments, pharmaceutical compositions are administered as needed, once per day, twice per day, three times per day, or four or more times per day. It is recognized by those skilled in the art that the particular dose, frequency, and duration of administration depends on a number of factors, including, without limitation, the biological activity desired, the condition of the subject, and tolerance for the pharmaceutical composition.

In certain embodiments, a pharmaceutical composition provided herein is administered for a period of continuous therapy. For example, a pharmaceutical composition provided herein can be administered over a period of days, weeks, months, or years.

Dosage amount, interval between doses, and duration of treatment can be adjusted to achieve a desired effect. In certain embodiments, dosage amount and interval between doses are adjusted to maintain a desired concentration of compound in a subject. For example, in certain embodiments, dosage amount and interval between doses are adjusted to provide plasma concentration of a salt or crystalline form thereof provided herein at an amount sufficient to achieve a desired effect. In certain of such embodiments the plasma concentration is maintained above the minimal effective concentration (MEC). In certain embodiments, pharmaceutical compositions provided herein are administered with a dosage regimen designed to maintain a concentration above the MEC for 10-90% of the time, between 30-90% of the time, or between 50-90% of the time.

### Compositions for Oral Administration

In certain embodiments, oral pharmaceutical dosage forms are either solid, gel or liquid. The solid dosage forms are tablets, capsules, granules, and bulk powders. Types of oral tablets include compressed, chewable lozenges and tablets which can be enteric coated, sugar coated or film coated. Capsules can be hard or soft gelatin capsules, while granules and powders can be provided in non-effervescent or effervescent form with the combination of other ingredients known to those skilled in the art.

In certain embodiments, the formulations are solid dosage forms, such as capsules or tablets. The tablets, pills, capsules, troches and other solid dosage forms can include any of the following ingredients, or compounds of a similar nature: a binder; a diluent; a disintegrating agent; a lubricant; a glidant; a sweetening agent; and a flavoring agent.

In certain embodiments, pharmaceutical compositions for oral administration are push fit capsules made of gelatin. Certain of such push fit capsules include one or more compounds provided herein in admixture with one or more fillers such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more compounds provided are be dissolved or suspended in suitable liquids, such as fatty oils, liquid paraffin, or liquid polyethylene glycols. In addition, stabilizers can be added.

In certain embodiments, pharmaceutical compositions are prepared for buccal administration. Certain of such pharmaceutical compositions are tablets or lozenges formulated in conventional manner. In some embodiments, the compositions are formulated as dissolvable films, such as those made with pullulan or described in the art (e.g., see U.S. Pat. Nos. 6,596,298, 7,067,116, 7,182,964 and 7,241,411).

Examples of binders for use in the compositions provided herein include microcrystalline cellulose, gum tragacanth, glucose solution, gum arabic, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include croscarmellose sodium, sodium starch glycolate, alginic acid, sodium alginate, corn starch, potato starch, bentonite, methylcellulose, agar and carboxymethylcellulose. Coloring agents include, for example, any of the approved certified water-soluble FD and C dyes, mixtures thereof; and water insoluble FD and C dyes suspended on alumina hydrate. Sweetening agents include sucrose, lactose, mannitol, xylitol and artificial sweetening agents such as saccharin. Flavoring agents include natural flavors extracted from plants such as fruits and synthetic blends of compounds which produce a pleasant sensation, such as, but not limited to peppermint and methyl salicylate, including spray dried natural and artificial flavors. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound can be provided in a composition that protects it from the acidic environment of the stomach. For example, the composition can be formulated in an enteric coating that maintains its integrity in the stomach and releases the active compound in the intestine. The composition also can be formulated in combination with an antacid or other such ingredient.

When the dosage unit form is a capsule, it can include, in addition to material of the above type, a liquid carrier such as a fatty oil. In addition, dosage unit forms can include various other materials which modify the physical form of the dosage unit, for example, coatings of sugar and other enteric agents. The compounds also can be administered as a component of an elixir, suspension, syrup, wafer, sprinkle or chewing gum. A syrup can include, in addition to the active compounds, sucrose as a sweetening agent and certain preservatives, dyes and colorings and flavors.

The active materials also can be mixed with other active materials which do not impair the desired action, or with materials that supplement the desired action, such as antacids, H2 blockers (acid reducers), and diuretics.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents also can be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and can include a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, gum arabic, gum tragacanth, xanthan gum, propylene glycol alginate, bentonite, and surfactants such as polyoxyethylene sorbitan monooleate. Suspending agents include xanthan gum, sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water-soluble FD and C dyes, and mixtures thereof. Flavoring agents include natural flavors extracted from plants such fruits, and synthetic blends of compounds which produce a pleasant taste sensation.

For a solid dosage form, the solution or suspension, e.g., in propylene carbonate, vegetable oils or triglycerides, can be encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, can be diluted with a sufficient quantity of a pharmaceutically acceptable liquid carrier, e.g., water, to be easily measured for administration.

Alternatively, liquid or semi-solid oral formulations can be prepared by dissolving or dispersing the salt or crystalline form thereof provided herein in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE 28,819 and 4,358,603. Briefly, such formulations include, but are not limited to, those including a compound provided herein, a dialkylated mono- or poly-alkylene glycol, including, but not limited to, 1,2-dimethoxy-methane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations can be coated as known by those of skill in the art in order to modify or sustain dissolution of the salt or crystalline form thereof described herein. Thus, for example, they can be coated with a conventional enterically digestible coating, such as phenylsalicylate, waxes and cellulose acetate phthalate.

Exemplary compositions can include fast-dissolving diluents such as mannitol, lactose, sucrose, and/or cyclodextrins. Also included in such formulations can be high molecular weight excipients such as celluloses and microcrystalline celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers also can be added for ease of fabrication and use.

In certain of such embodiments, a pharmaceutical composition for oral administration is formulated by combining one or more salt and crystalline forms thereof provided herein with one or more pharmaceutically acceptable carriers. Certain of such carriers enable compounds provided herein to be formulated in dosage forms, such as tablets, pills, dragees, capsules, liquids, gels, syrups, slurries and suspensions, for oral ingestion by a subject. In certain embodiments, pharmaceutical compositions for oral use are obtained by mixing one or more salt and crystalline forms thereof provided herein and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross linked polyvinyl pyrrolidone, agar, or alginic acid or a salt thereof, such as sodium alginate) are added.

In certain embodiments, dragee cores are provided with coatings. In certain of such embodiments, concentrated sugar solutions can be used, which can optionally include gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to tablets or dragee coatings.

In certain embodiments, a daily dosage regimen for a subject includes an oral dose of between 0.1 μg and 2000 mg of a salt or crystalline form thereof provided herein. In certain embodiments, a daily dosage regimen for a subject includes an oral dose of between 1 μg and 500 mg of a salt or crystalline form thereof provided herein. In certain embodiments, a daily dosage regimen for a subject includes an oral dose of between 10 μg and 100 mg of a salt or crystalline form thereof provided herein. In certain embodiments, a daily dosage regimen for a subject includes an oral dose selected from among 0.01, 0.05, 0.075, 0.1, 0.25, 0.5, 0.75, 1, 2, 2.5, 3, 4, 5, 6, 7, 8, 9, 10, 15, 20, 25, 50, 75, 100, 125, 150, 175, 180, 190, 200, 225, 250, 300, 400, 500, 750, 800, 850, 900, 950 and 1000 milligrams of a salt or crystalline form thereof provided herein. In certain embodiments, a daily dosage regimen is administered as a single daily dose. In certain embodiments, a daily dosage regimen is administered as two, three, four, or more than four doses.

### Injectables, Solutions and Emulsions

In certain embodiments, the pharmaceutical composition is prepared for transmucosal administration. In certain of such embodiments penetrants appropriate to the barrier to be permeated are used in the formulation. Such penetrants are generally known in the art.

Parenteral administration, generally characterized by injection, either subcutaneously, intramuscularly or intravenously also is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol, mannitol, 1,3-butanediol, Ringer's solution, an isotonic sodium chloride solution or ethanol. In addition, if desired, the pharmaceutical compositions to be administered also can include minor amounts of non-toxic auxiliary substances such as wetting or emulsifying agents, pH buffering agents, stabilizers, solubility enhancers, and other such agents, such as for example, mono- or diglycerides, fatty acids, such as oleic acid, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) also is contemplated herein. Briefly, a salt or crystalline form thereof provided herein is dispersed in a solid inner matrix, e.g., polymethyl-methacrylate, polybutylmethacrylate, plasticized or unplasticized polyvinylchloride, plasticized nylon, plasticized polyethylene-terephthalate, natural rubber, polyisoprene, polyisobutylene, polybutadiene, polyethylene, ethylene-vinylacetate copolymers, silicone rubbers, polydimethyl-siloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxy-ethanol copolymer, that is insoluble in body fluids. The salt or crystalline form thereof diffuses through the outer polymeric membrane in a release rate controlling step. The percentage of active compound included in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions including thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Anti-oxidants include sodium bisulfate. Local anesthetics include procaine hydrochloride. Suspending and dispersing agents include sodium carboxymethylcelluose, hydroxy-propyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the subject or animal as is known in the art.

The unit dosage parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution including an active compound is an effective mode of administration. Another embodiment is a sterile aqueous or oily solution or suspension including an active material injected as necessary to produce the desired pharmacological effect.

Injectables are designed for local and systemic administration. Typically, a therapeutically effective dosage is formulated to include a concentration of at least about 0.1% w/w up to about 90% w/w or more, in some embodiments more than 1% w/w, of the active compound to the treated tissue(s). The active ingredient can be administered at once, or can be divided into a number of smaller doses to be administered at intervals of time. The precise dosage and duration of treatment is a function of the tissue being treated and can be determined empirically using known testing protocols or by extrapolation from in vivo or in vitro test data. It is to be noted that concentrations and dosage values also can vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of formulations provided herein.

The salts and crystalline forms thereof can be formulated in any suitable vehicle or form. For example, they can be in micronized or other suitable form and/or can be derivatized to produce a more soluble active product or to produce a prodrug or for other purposes. The form of the resulting mixture depends upon a number of factors, including, for example, an intended mode of administration and the solubility of the salt and polymorphs thereof in the selected carrier or vehicle. The effective concentration is sufficient for ameliorating the symptoms of the condition and can be empirically determined.

In certain embodiments, a pharmaceutical composition is prepared for administration by injection wherein the pharmaceutical composition includes a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers and/or suspending agents. Certain pharmaceutical compositions for injection are presented in unit dosage form, e.g., in ampules or in multi dose containers. Certain pharmaceutical compositions for injection are suspensions, solutions or emulsions in oily or aqueous vehicles, and can include formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions can include substances that increase the viscosity of the suspension, such as sodium carboxymethyl cellulose, sorbitol, or dextran. Optionally, such suspensions also can include suitable stabilizers or agents that increase the solubility of the compounds to allow for the preparation of highly concentrated solutions.

In certain embodiments, the pharmaceutical compositions provided are administered by continuous intravenous infusion. In certain of such embodiments, from 0.01 g to 500 mg of the composition is administered per day.

### Lyophilized Powders

Of interest herein also are lyophilized powders, which can be reconstituted for administration as solutions, emulsions and other mixtures. They also can be reconstituted and formulated as solids or gels.

The sterile, lyophilized powder is prepared by dissolving a salt or crystalline form thereof provided herein in a suitable solvent. The solvent can include an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent also can include a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. In some embodiments, each vial includes a single dosage of from 10 μg to 1000 mg. In another embodiment, each vial includes a single dosage of from 100 μg to 500 mg. In another embodiment, each vial includes a single dosage of from 0.1 mg to 50 mg. In another embodiment, each vial includes a single dosage of from 0.5 mg to 20 mg. In another embodiment, each vial includes a single dosage of 1 mg, 2 mg, 3 mg, 4 mg, 5 mg, 6 mg, 7 mg, 8 mg, 9 mg or 10 mg. In another embodiment, each vial includes multiple dosages of the compound salt or crystalline form thereof described herein. The lyophilized powder can be stored under appropriate conditions, such as at about 4° C. to room temperature.

Reconstitution of this lyophilized powder with water for injection provides a formulation for use in parenteral administration. For reconstitution, about 1 mg to 50 mg is added per mL of sterile water or other suitable carrier. In some embodiments, 5 mg to 35 mg is added per mL of sterile water or other suitable carrier. In other embodiments, 10 mg to 30 mg of lyophilized powder is added per mL of sterile water or other suitable carrier. The precise amount depends upon the selected compound. Such amount can be empirically determined.

### Methods of Treatment Using Salt of the Compound of Formula (I), Crystalline Forms Thereof and Compositions Thereof

Methods of use of the salt and crystalline forms thereof and compositions provided herein also are provided. The methods include the treatment, prevention, or amelioration of one or more of the diseases, disorders, or medical conditions referred to hereinabove, such as eye diseases associated with, but not limited to, inflammation and/or vascular proliferation as described herein or fibrotic diseases and disorders, including those as described herein. In certain embodiments, provided herein are methods of treating a subject by administering a salt and polymorphs thereof provided herein. In certain embodiments, a subject is treated prophylactically to reduce or prevent the occurrence of a condition.

Administration of salts of the compound of Formula (I) and crystalline forms thereof or any composition(s) comprising same to subjects can be by any of the accepted modes for enteral administration such as oral or rectal, or by parenteral administration such as subcutaneous, intramuscular, intravenous and intradermal routes. Injection can be bolus or via constant or intermittent infusion. The active compound is typically included in a pharmaceutically acceptable carrier, excipient and/or diluent and in an amount sufficient to deliver to the patient a therapeutically effective dose.

The salts of the compound of Formula (I) and crystalline forms thereof may be formulated into a composition for administering to a subject in need thereof. For example, in one embodiment, one or more salt of the compound of Formula (I) and crystalline forms thereof may be combined with one or more excipients into a solid formulation for administering to a subject in need thereof. In other embodiments, one or more salt of the compound of Formula (I) and crystalline forms thereof may be formulated into a liquid composition for administering to a subject in need thereof. In some such embodiments, the one or more salt of the compound of Formula (I) and crystalline forms thereof may be dissolved in one or more liquid solvents to form a solution for administering to a subject in need thereof. In other such embodiments, the one or more salt of the compound of Formula (I) and crystalline forms thereof may be dispersed in one or more liquids to form a suspension for administering to a subject in need thereof.

The compositions provided herein can be used in the treatment of a variety of conditions. For example, a composition comprising one or more salt of the compound of Formula (I) and crystalline forms thereof can be used to treat a condition including, but not limited to, eye disease or disorders such as diabetic retinopathy, corneal edema, anterior and posterior uveitis, pterygium, corneal diseases, dry eye, conjunctivitis, allergy- and laser-induced exudation, non-age related macular degeneration, macular edema, age-related macular degeneration and/or ocular von Hippel-Lindau disease.

In another embodiment, a composition comprising one or more salts of the compound of Formula (I) and crystalline forms thereof can be used to treat a condition including, but not limited to, corneal diseases, which may be caused by infection from a microbe or microorganism. The microbe or microorganism may be selected from any of the groups comprising bacteria, viruses, fungi, amoebas, and parasites.

In another embodiment, a composition comprising one or more salt of the compound of Formula (I) and crystalline forms thereof can be used to treat a condition including, but not limited to, eye diseases associated with inflammation and/or vascular proliferation disease.

In another embodiment, a composition comprising one or more salt of the compound of Formula (I) and crystalline forms thereof can be used to reduce at least one, but not limited to, the level of ICAM-1 mRNA in the retina of the subject; the level of VEGF mRNA in the retina of the subject; and the number of leukocytes in the retina of the subject.

In another embodiment, a composition comprising one or more salt of the compound of Formula (I) and crystalline forms thereof can be used for treating a disease or condition associated with fibrosis. Such diseases may include, for example, fibrotic skin disorders, such as keloids, hypertrophic scars and scleroderma; lung disease, such as pulmonary fibrosis; heart disease, such as heart failure due to ischaemic heart disease, valvular heart disease and hypertensive heart disease, diabetic cardiomyopathy and hypertension; and kidney disease, such as progressive kidney disease, due to, glomerulonephritis and diabetic nephropathy and cirrhosis of the liver. In a preferred embodiment, the disease or condition is diabetic heart disease or diabetic kidney disease. In a further preferred embodiment, the disease or condition is diabetic cardiomyopathy. In some embodiments, the kidney disease may include, but is not limited to, a progressive glomerular kidney disease including without limitation diabetic nephropathy (e.g., as a consequence of Type I or Type II diabetes or systemic lupus), primary glomerulonephritis (e.g., membranous nephropathy, focal segmental glomerulosclerosis, membranoproliferative glomerulonephritis, diffuse proliferative glomerulonephritis, membranous focal segmental glomerulosclerosis) or secondary glomerulonephritis (e.g., diabetic nephropathy, ischemic nephropathy). In some embodiments, the kidney disease may include progressive kidney diseases with origins primarily in the tubulointerstitium. In some embodiments, the kidney disease may include, e.g., chronic interstitial nephritis, autosomal dominant tubulointerstitial fibrosis, or reflux nephropathy.

In another embodiments, a kit for treating an eye disease associated with inflammation and/or vascular proliferation including: at least one salt of the compound of Formula (I) and crystalline forms thereof; and instructions for administering the same to a subject to treat the eye disease.

In another embodiments, a kit for treating a disease or condition associated with fibrosis including: at least one salt of the compound of Formula (I) and crystalline forms thereof; and instructions for administering the same to a subject to treat the disease or condition associated with fibrosis.

In certain embodiments, provided are methods for treating a subject by administering a composition comprising at least one salt of the compound of Formula (I) or crystalline forms thereof.

In certain embodiments, provided are methods for treating a subject by administering a composition comprising salts of the compound of Formula (I) and polymorphs thereof by reducing at least one of: the level of ICAM-1 mRNA in the retina of the subject; the level of VEGF mRNA in the retina of the subject; and the number of leukocytes in the retina of the subject.

### Methods of Use of Crystalline Forms of the Compound of Formula (I) and Compositions Thereof

In certain embodiments, one or more salt of the compound of Formula (I) and crystalline forms thereof provided herein can be co-administered with one or more other therapeutic agents. In certain embodiments, such one or more other therapeutic agents are designed to treat the same disease or condition as the one or more compounds or pharmaceutical compositions provided herein. In certain embodiments, such one or more other therapeutic agents are designed to treat a different disease or condition as the one or more compounds or compositions provided herein. In certain embodiments, such one or more other therapeutic agents are designed to treat an undesired effect of one or more compounds or compositions provided herein. In certain embodiments, one or more compounds or compositions provided herein is co-administered with another therapeutic agent to treat an undesired effect of that other agent.

In using the salts of the compound of Formula (I) and crystalline forms thereof they can be administered in any form or mode which makes the compound bioavailable. One skilled in the art of preparing formulations can readily select the proper form and mode of administration depending upon the particular characteristics of the compound selected, the condition to be treated, the stage of the condition to be treated and other relevant circumstances. See Remington's Pharmaceutical Sciences, 19th edition, Mack Publishing Co. (1995) for further information.

The salts of the compound of Formula (I) and crystalline forms thereof can be administered alone or in the form of a pharmaceutical composition in combination with a pharmaceutically acceptable carrier, diluent, adjuvant and/or excipient.

The salts of the compound of Formula (I) and crystalline forms thereof are, however, typically used in the form of pharmaceutical compositions which are formulated depending on the desired mode of administration. Suitable compositions for use in accordance with the methods of the present disclosure may be prepared according to methods and procedures that are known to those of ordinary skill in the art and accordingly may include a pharmaceutically acceptable carrier, excipient, diluent and/or adjuvant. As such in a further embodiment the present disclosure provides a pharmaceutical composition including a salt of the compound of Formula (I) and crystalline forms thereof and a pharmaceutically acceptable carrier, diluent, adjuvant or excipient.

In certain embodiments, compounds or compositions provided herein and one or more other therapeutic agents are administered at the same time. In some embodiments, compounds or compositions provided herein and one or more other therapeutic agents are administered at the different times. In certain embodiments, compounds or compositions provided herein and one or more other therapeutic agents are prepared together in a single formulation. In certain embodiments, compounds or compositions provided herein and one or more other therapeutic agents are prepared separately.

In another embodiment a pharmaceutical pack or kit comprising one or more containers filled with one or more of the ingredients of the pharmaceutical compositions. In such a pack or kit can be found a container having a unit dosage of the agent(s). The kits can include a composition comprising an effective agent either as concentrates (including lyophilized compositions), which can be diluted further prior to use or they can be provided at the concentration of use, where the vials may include one or more dosages. Conveniently, in the kits, single dosages can be provided in sterile vials so that the physician can employ the vials directly, where the vials will have the desired amount and concentration of agent(s). Associated with such container(s) can be various written materials such as instructions for use, or a notice in the form prescribed by a governmental agency regulating the manufacture, use or sale of pharmaceuticals or biological products, which notice reflects approval by the agency of manufacture, use or sale for human administration.

The salts and crystalline forms described herein may be used or administered in combination with one or more additional drug(s) for the treatment of the disorder/diseases mentioned. The components can be administered in the same formulation or in separate formulations or compositions. If administered in separate formulations or compositions, the salts and crystalline forms thereof of the disclosure or the formulations or compositions comprising same may be administered sequentially or simultaneously with the other drug(s).

In addition to being able to be administered in combination with one or more additional drugs, the salts of the compound of Formula (I) and crystalline forms thereof may be used in a combination therapy. When this is done the compounds are typically administered in combination with each other. Thus, one or more of the salts of the compound of Formula (I) and crystalline forms thereof of the disclosure may be administered either simultaneously (as a combined preparation) or sequentially in order to achieve a desired effect. This is especially desirable where the therapeutic profile of each salt of the compound of Formula (I) and crystalline forms thereof is different such that the combined effect of the two drugs provides an improved therapeutic result. Administration may be systemic, regional or local.

Pharmaceutical compositions of the disclosed salts of the compound of Formula (I) and crystalline forms thereof may be administered by standard routes. In general, the compositions may be administered by the parenteral (e.g., intravenous, intraspinal, subcutaneous or intramuscular), oral or topical route. The particular route of administration to be used in any given circumstance will depend on a number of factors, including the nature of the condition to be treated, the severity and extent of the condition, the required dosage of the particular compound to be delivered and the potential side-effects of the compound.

Pharmaceutical compositions of the disclosed salts of the compound of Formula (I) and crystalline forms thereof for parenteral injection comprise pharmaceutically acceptable sterile aqueous or nonaqueous solutions, dispersions, suspensions or emulsions as well as sterile powders for reconstitution into sterile injectable solutions or dispersions just prior to use.

In using the salts of the compound of Formula (I) and crystalline forms thereof, one skilled in the art may prepare pharmaceutical acceptable excipient compositions utilizing Remington's Pharmaceutical Sciences, 19th edition, Mack Publishing Co. (1995).

The topical compositions of the present disclosure, comprise an active ingredient together with one or more acceptable carriers, and optionally any other therapeutic ingredients. Compositions suitable for topical administration include liquid or semi-liquid preparations suitable for penetration through the skin to the site of where treatment is required, such as liniments, lotions, creams, powders, patches, sprays, inhalers, ointments or pastes, and drops suitable for administration to the eye, ear or nose. The active ingredient is mixed under sterile conditions with a pharmaceutically acceptable carrier and any needed preservatives, buffers, or propellants which may be required.

Drops according to the present disclosure may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by: autoclaving or maintaining at 90-100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present disclosure include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present disclosure are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The amount of compound administered will preferably treat and reduce or alleviate the condition. A therapeutically effective amount can be readily determined by an attending diagnostician by the use of conventional techniques and by observing results obtained under analogous circumstances. In determining the therapeutically effective amount a number of factors are to be considered including but not limited to, the species of animal, its size, age and general health, the specific condition involved, the severity of the condition, the response of the patient to treatment, the particular compound administered, the mode of administration, the bioavailability of the preparation administered, the dose regime selected, the use of other medications and other relevant circumstances.

One skilled in the art would be able, by routine experimentation, to determine an effective, non-toxic amount of the compound which would be required to treat applicable diseases and conditions. Generally, a typical and effective dosage is expected to be in the range of about 0.01 to about 1000 mg per kilogram of body weight per day, typically of about 0.1 to about 100 mg per kilogram of body weight per day, even more typically of about 1 to about 10 mg per kilogram of body weight per day, and further of about 5 mg per kilogram of body weight per day. Small doses (0.01-1 mg/kg per day) may be administered initially, followed by increasing doses up to about 1000 mg/kg per day. In the event that the response in a subject is insufficient at such doses, even higher doses (or effective higher doses by a different, more localized delivery route) may be employed to the extent patient tolerance permits. A more preferred dosage will be in the range from 0.1 to 300 mg per kilogram of body weight per day, more preferably from 0.1 to 100 mg per kilogram of body weight per day. A suitable dose can be administered in multiple sub-doses per day.

Typically, in therapeutic applications, the treatment would be for the duration of the disease state.

Further, it will be apparent to one of ordinary skill in the art that the optimal quantity and spacing of individual dosages such as the number of doses of the composition given per day for a defined number of days will be determined by the nature and extent of the disease state being treated, the form, route and site of administration, and the nature of the particular individual being treated.

Disclosed herein are the following numbered embodiments:

Embodiment 1: A crystalline form of a pharmaceutically acceptable salt of the compound of Formula (I):

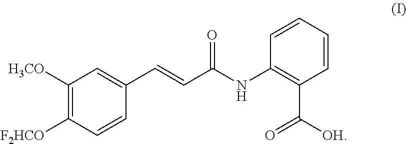 [SMILES: COc1cc(/C=C/C(=O)Nc2ccccc2C(=O)O)ccc1OC(F)F]

Embodiment 2: The crystalline form of embodiment 1, wherein the pharmaceutically acceptable salt is selected from the group consisting of: a sodium salt, a potassium salt, a zinc salt, a magnesium salt, and a calcium salt.

Embodiment 3: The crystalline form of embodiment 1, wherein the pharmaceutically acceptable salt is selected from the group consisting of: a choline salt, an L-lysine salt, a tert-butylamine salt, and a diethanolamine salt.

Embodiment 4: The crystalline form of embodiment 3, wherein the pharmaceutically acceptable salt is an L-lysine salt.

Embodiment 5: The crystalline form of embodiment 4, wherein the crystalline form exhibits an X-ray powder diffraction pattern having at least three characteristic peaks, wherein the three characteristic peaks are selected from the group consisting of peaks at 4.23, 7.92, 10.59, 14.44, 15.25, 15.50, 19.32, 19.87, 21.30, 23.41, 23.94, 24.42, 25.30, 25.81, 26.00 degrees 2θ (±0.2 degrees 2θ).

Embodiment 6: The crystalline form of embodiment 4, wherein the crystalline form exhibits an X-ray powder diffraction pattern having at least four characteristic peaks, wherein the four characteristic peaks are selected from the group consisting of peaks at 4.23, 7.92, 10.59, 14.44, 15.25, 15.50, 19.32, 19.87, 21.30, 23.41, 23.94, 24.42, 25.30, 25.81, 26.00 degrees 2θ (±0.2 degrees 2θ).

Embodiment 7: The crystalline form of embodiment 4, wherein the crystalline form exhibits an X-ray powder diffraction pattern having at least five characteristic peaks, wherein the five characteristic peaks are selected from the group consisting of peaks at 4.23, 7.92, 10.59, 14.44, 15.25, 15.50, 19.32, 19.87, 21.30, 23.41, 23.94, 24.42, 25.30, 25.81, 26.00 degrees 2θ (±0.2 degrees 2θ).

Embodiment 8: The crystalline form of embodiment 4, wherein the crystalline form exhibits an X-ray powder diffraction pattern as depicted in FIG. 1.

Embodiment 9: The crystalline form of any one of embodiments 4, 5, 6, 7, and 8, wherein the crystalline form has a melting point of about 197-203° C.

Embodiment 10: The crystalline form of embodiment 3, wherein the pharmaceutically acceptable salt is a tert-butylamine salt.

Embodiment 11: The crystalline form of embodiment 10, wherein the crystalline form exhibits an X-ray powder diffraction pattern having at least three characteristic peaks, wherein the three characteristic peaks are selected from the group consisting of peaks at 13.59, 13.75, 17.93, 18.36, 18.44, 19.39, 20.26, 20.77, 21.22, 21.78, 21.84, 22.82, 23.34, 23.91, 26.12 degrees 2θ (±0.2 degrees 2θ).

Embodiment 12: The crystalline form of embodiment 10, wherein the crystalline form exhibits an X-ray powder diffraction pattern having at least four characteristic peaks, wherein the four characteristic peaks are selected from the group consisting of peaks at 13.59, 13.75, 17.93, 18.36, 18.44, 19.39, 20.26, 20.77, 21.22, 21.78, 21.84, 22.82, 23.34, 23.91, 26.12 degrees 2θ (±0.2 degrees 2θ).

Embodiment 13: The crystalline form of embodiment 10, wherein the crystalline form exhibits an X-ray powder diffraction pattern having at least five characteristic peaks, wherein the five characteristic peaks are selected from the group consisting of peaks at 13.59, 13.75, 17.93, 18.36, 18.44, 19.39, 20.26, 20.77, 21.22, 21.78, 21.84, 22.82, 23.34, 23.91, 26.12 degrees 2θ (±0.2 degrees 2θ).

Embodiment 14: The crystalline form of embodiment 10, wherein the crystalline form exhibits an X-ray powder diffraction pattern as depicted in FIG. 3.

Embodiment 15: The crystalline form of any one of embodiments 10, 11, 12, 13, or 14, wherein the crystalline form has a melting point of about 196-224° C.

Embodiment 16: A pharmaceutical composition comprising a crystalline form of any one of embodiments 1, 2, 3, 4, 5, 6, 7, 8, 9, 10, 11, 12, 13, 14, 15, and 16 and a pharmaceutically acceptable excipient.

Embodiment 17: The L-lysine salt of the compound of Formula (I):

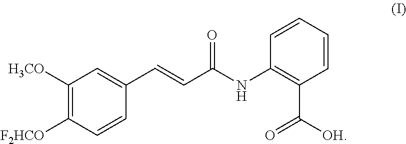 [SMILES: COc1cc(/C=C/C(=O)Nc2ccccc2C(=O)O)ccc1OC(F)F]

Embodiment 18: The tert-butylamine salt of the compound of Formula (I):

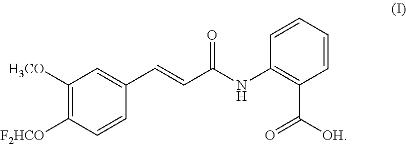 [SMILES: COc1cc(/C=C/C(=O)Nc2ccccc2C(=O)O)ccc1OC(F)F]

Embodiment 19: A method of treating an eye disease comprising administering to a subject a therapeutically effective amount of the crystalline form of any one of embodiments 1, 2, 3, 4, 5, 6, 7, 8, 9, 10, 11, 12, 13, 14, 15, and 16.

Embodiment 20: The method of embodiment 19, wherein the eye disease selected from the group comprising diabetic retinopathy, proliferative vitreoretinopathy corneal edema, anterior and posterior uveitis, pterygium, corneal diseases, dry eye, conjunctivitis, allergy- and laser-induced exudation, non-age related macular degeneration, macular edema, age-related macular degeneration and ocular von Hippel-Lindau disease.

Embodiment 21: A method of reducing at least one of: the level of ICAM-1 mRNA in the retina of the subject; the level of VEGF mRNA in the retina of the subject; and the number of leukocytes in the retina of the subject, the method comprising administering to a subject a therapeutically effective amount of the crystalline form of any one of embodiments 1, 2, 3, 4, 5, 6, 7, 8, 9, 10, 11, 12, 13, 14, and 15, or the pharmaceutical composition of embodiment 16.

Embodiment 22: A method of treating a disease or condition associated with fibrosis, the method comprising administering to a subject a therapeutically effective amount of the crystalline form of any one of embodiments 1 to 16.

Embodiment 23: The method of embodiment 22, wherein the disease or disorder associated with fibrosis is selected from the group consisting of: a fibrotic skin disorder, a lung disease, a heart disease, and a kidney disease.

Embodiment 24: The method of embodiment 23, wherein the fibrotic skin disorder is selected from the group consisting of: keloids, hypertrophic scars and scleroderma.

Embodiment 25: The method of embodiment 23, wherein the lung disease is pulmonary fibrosis.

Embodiment 26: The method of embodiment 23, wherein the heart disease is selected from the group consisting of: heart failure due to ischaemic heart disease, diabetic heart disease, valvular heart disease, hypertensive heart disease, diabetic cardiomyopathy, and hypertension.

Embodiment 27: The method of embodiment 23, wherein the kidney disease is selected from the group consisting of: progressive glomerular kidney disease and diabetic kidney disease.

Embodiment 28: The method of embodiment 27, wherein the progressive kidney disease is primary glomerulonephritis or secondary glomerulonephritis

Embodiment 29: The method of embodiment 28, wherein the primary glomerulonephritis is selected from the group consisting of: membranous nephropathy, focal segmental glomerulosclerosis, membranoproliferative glomerulonephritis, diffuse proliferative glomerulonephritis, and membranous focal segmental glomerulosclerosis.

Embodiment 30: The method of embodiment 28, wherein the secondary glomerulonephritis is diabetic nephropathy or ischemic nephropathy.

Embodiment 31: The method of embodiment 23, wherein the kidney disease is a progressive kidney diseases with origins primarily in the tubulointerstitium.

Embodiment 32: The method of Claim 31, wherein the kidney disease is interstitial nephritis, autosomal dominant tubulointerstitial fibrosis, or reflux nephropathy.

Embodiment 33: The method of any one of embodiments 19, 20, and 21, wherein the crystalline form or composition is administered orally.

Embodiment 34: The method of any one of embodiments 19, 20, and 21, wherein the crystalline form or composition is administered to the eye.

Embodiment 35: A method of treating an eye disease comprising administering to a subject a therapeutically effective amount of the compound of any one of embodiments 17 and 18.

Embodiment 36: The method of embodiment 35, wherein the eye disease selected from the group comprising diabetic retinopathy, proliferative vitreoretinopathy corneal edema, anterior and posterior uveitis, pterygium, corneal diseases, dry eye, conjunctivitis, allergy- and laser-induced exudation, non-age related macular degeneration, macular edema, age-related macular degeneration and ocular von Hippel-Lindau disease.

Embodiment 37: A method of reducing at least one of: the level of ICAM-1 mRNA in the retina of the subject; the level of VEGF mRNA in the retina of the subject; and the number of leukocytes in the retina of the subject, the method comprising administering to a subject a therapeutically effective amount of the compound of any one of embodiments 17 and 18.

Embodiment 38: A method of treating a disease or condition associated with fibrosis, the method comprising administering to a subject a therapeutically effective amount of the compound of embodiment 17 or 18.

Embodiment 39: The method of embodiment 38, wherein the disease or disorder associated with fibrosis is selected from the group consisting of: a fibrotic skin disorder, a lung disease, a heart disease, and a kidney disease.

Embodiment 40: The method of embodiment 39, wherein the fibrotic skin disorder is selected from the group consisting of: keloids, hypertrophic scars and scleroderma.

Embodiment 41: The method of embodiment 39, wherein the lung disease is pulmonary fibrosis.

Embodiment 42: The method of embodiment 39, wherein the heart disease is selected from the group consisting of: heart failure due to ischaemic heart disease, diabetic heart disease, valvular heart disease, hypertensive heart disease, diabetic cardiomyopathy, and hypertension.

Embodiment 43: The method of embodiment 39, wherein the kidney disease is selected from the group consisting of: progressive glomerular kidney disease and diabetic kidney disease.

Embodiment 44: The method of embodiment 42, the progressive glomerular kidney disease is primary glomerulonephritis or secondary glomerulonephritis.

Embodiment 45: The method of embodiment 44, wherein the primary glomerulonephritis is selected from the group consisting of: membranous nephropathy, focal segmental glomerulosclerosis, membranoproliferative glomerulonephritis, diffuse proliferative glomerulonephritis, and membranous focal segmental glomerulosclerosis.

Embodiment 46: The method of embodiment 44, wherein the secondary glomerulonephritis is diabetic nephropathy or ischemic nephropathy.

Embodiment 47: The method of embodiment 39, wherein the kidney disease is a progressive kidney diseases with origins primarily in the tubulointerstitium.

Embodiment 48: The method of Claim 47, wherein the kidney disease is interstitial nephritis, autosomal dominant tubulointerstitial fibrosis, or reflux nephropathy.

Embodiment 49: The method of any one of embodiments 35 to 48, wherein the crystalline form or composition is administered orally.

Embodiment 50: The method of any one of embodiments 35 to 48, wherein the crystalline form or composition is administered to the eye.

### EXAMPLES

Additional embodiments are disclosed in further detail in the following examples, which are not in any way intended to limit the scope of the claims. X-ray Powder Diffraction (XRPD)

XRPD analysis was carried out on a PANalytical X'pert pro with PIXcel detector (128 channels), scanning the samples between 3 and 35° 2θ. The material was gently ground to release any agglomerates and loaded onto a multi-well plate with Kapton or Mylar polymer film to support the sample. The multi-well plate was then placed into the diffractometer and analyzed using Cu K radiation (α₁ λ=1.54060 Å; α₂=1.54443 Å; β=1.39225 Å; α₁: α₂ ratio=0.5) running in transmission mode (step size 0.0130° 2θ, step time 18.87 s) using 40 kV/40 mA generator settings. Data were visualized and images generated using the HighScore Plus 4.7 desktop application (PANalytical, 2017).

Alternatively, samples were analyzed on a Rigaku MiniFlex 600 with a D/tex detector. The optics were equipped with 5° Söller slits on both the incident and diffracted beam with a 1.250 DS, 8 mm SS, and 0.15 micron Ni KO filter. Scans were taken from 3 to 40° 2θ with 0.02°/step at 10° 2θ/min for powders or 3° 2θ/min for dropcast films. Thermogravimetric Analysis (TG)

Approximately, 5-10 mg of material was added into a pre-tared open aluminum pan and loaded into a TA Instruments Discovery SDT 650 Auto—Simultaneous DSC and held at room temperature. The sample was then heated at a rate of 10° C./min from 30° C. to 400° C. during which time the change in sample weight was recorded along with the heat flow response (DSC). Nitrogen was used as the sample purge gas, at a flow rate of 200 cm³/min.

### Differential Thermal Analysis (DTA)

Approximately, 5 mg of material was weighed into an open aluminium pan and loaded into a simultaneous thermogravimetric/differential thermal analyser (TG/DTA) and held at room temperature. The sample was then heated at a rate of 10° C./min from 20° C. to 300° C. during which time the change in sample weight was recorded along with any differential thermal events (DTA). Nitrogen was used as the purge gas, at a flow rate of 300 cm³/min.

### Dynamic Vapor Sorption (DVS)

Approximately, 10-20 mg of sample was placed into a mesh vapor sorption balance pan and loaded into a DVS Intrinsic dynamic vapor sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH. Two cycles were performed. The weight change during the sorption/desorption cycles were plotted, allowing for the hygroscopic nature of the sample to be determined. XRPD analysis was then carried out on any solid retained.

### Measurement of the Approximate Solubility

All samples were prepared at 20 mg/mL. The initial pH of each sample was recorded and adjusted as required using 0.1 M NaOH and 0.1 M HCl. The samples were agitated at 37° C. for 3 hours. The samples were collected, and the pH recorded and adjusted where required. The samples were filtered via centrifugation and the filtered mother liquors analyzed by HPLC.

HPLC conditions were as follows: Column: Waters Sunfire C18, 150×4.6 mm 3.5 m; Column Temperature: 25° C.; Autosampler Temperature: Ambient; UV wavelength: 220 nm; Injection Volume: 10 μL; Flow Rate: 1.0 mL/min; Mobile Phase A: 0.05% TFA in H₂O; Mobile Phase B: 0.05% TFA in CAN.

HPLC gradient program were as follows:

| Time (mins) | Solvent B [%] |
|---|---|
| 0 | 5 |
| 15 | 55 |
| 20 | 60 |
| 26 | 95 |
| 29 | 95 |
| 29.1 | 5 |
| 36 | 5 |

### Dissolution Studies

Discs for the intrinsic dissolution evaluation were prepared by weighing approximately 175 mg of each batch and compressing with a small hydraulic press in a 13 mm Wood's die for 3 minutes at a force of approximately 3000 lbs. The surface of each disc was examined carefully to ensure that it was uniform, smooth and free from loose powder.

The dissolution experiments were conducted by attaching the Wood's die containing the disc, to the matching shafts on an Erweka DT6 dissolution apparatus. The discs were lowered into the media (500 mL at 37° C.) and immediately rotated at 100 rpm. Duplicate samples of the media (0.2 mL) were removed prior to immersion of the disc and then at 2, 5, 10, 15, 20, 25, 30, 60 and 90 minutes after the experiment was initiated. Sampling was discontinued if substantial dissolution was visible or if the disc disintegrated in the media.

At the end of each experiment, the discs were removed from the media and examined to ensure that the surface of the discs remained uniform and to qualitatively assess the extent of dissolution over the course of the experiment. Media samples were centrifuged (10000 rpm×3 min) and a small aliquot (50 μL) removed, 5-fold diluted with 50% aqueous acetonitrile prior to HPLC analysis.

### Example 1

### Preparation of Crystalline Form of the L-Lysine Salt of the Compound of Formula (I) (“Crystalline Salt A”)

The compound of Formula (I) was dissolved in a minimum amount of methanol. L-lysine (1.05 equivalents) was then added, and the sample temperature cycled between ambient temperature and 40° C. in 4-hour cycles for ca. 72 hours. After this time, a single anti-solvent, toluene, was added to the mixture and the samples stored at 2-8° C. to promote precipitation. Other solvent/anti-solvent systems were used, including (i) 2-propanol/heptane, (ii) acetonitrile/toluene, and (iii) ethanol/heptane. The L-lysine salt of the compound of Formula (I) was isolated as crystalline material via centrifugation filtration. Where no crystalline material was obtained, the solvents were allowed to evaporate under ambient conditions. The L-lysine salt of the compound of Formula (I) was obtained as crystalline material. The observed solids were dried under vacuum at 40° C. for ca. 6 hours.

### Example 2

### Preparation of Crystalline Form of the Tert-Butylamine Salt of the Compound of Formula (I) (“Crystalline Salt B”)

The compound of Formula (I) was dissolved in a minimum amount of a single solvent selected from 2-propanol, acetonitrile, ethyl acetate, and isopropyl acetate. tert-Butylamine (1.05 equivalents) was then added and the sample temperature cycled between ambient temperature and 40° C. in 4-hour cycles for ca. 72 hours. After this time, the tert-butylamine salt of the compound of Formula (I) was obtained as crystalline material by filtration. Where no crystalline material was obtained, a single anti-solvent, selected from heptane and toluene, was added to the mixture and the solvents allowed to evaporate under ambient conditions. After this time the tert-butylamine salt of the compound of Formula (I) was obtained as crystalline material. The observed solids were dried under vacuum at 40° C. for ca. 6 hours.

### Example 2

### Additional Preparation of Crystalline Form I of the Tert-Butylamine Salt of the Compound of Formula (I)

The compound of Formula (I) (0.83 mmol) was dissolved in 7 mL of ethyl acetate at 40° C. with stirring. (0.87 mmol) tert-butylamine was added to the solution of the compound of Formula (I), which resulted in immediate precipitation of the tert-butylamine salt. The mixture was annealed for 72 hours by temperature cycling between 25° C. and 40° C. Vacuum filtration of the solid followed by vacuum drying at 40° C. gave a crystalline white powder which was identified as the tert-butylamine salt Form I.

### Example 3

### Seeded Recrystallization Preparation of Form I of the Tert-Butylamine Salt of the Compound of Formula (I)

Crystalline Salt B (12.72 wt %) in 80/20 methanol/ethyl acetate was prepared at 20° C. The temperature was then ramped to 60° C. at 1° C./min and held for 1 hour. Next, the mixture was cooled to 50° C. at 0.3° C./min and held for at that temperature for 75 minutes. The mixture was seeded with 2 wt % seeds of Form I and then cooled to 10° C. at 0.08° C./min and held for 1 hour. Ethyl acetate was then added over a period of 2 hours until the ratio of methanol to ethyl acetate reached 20/80. The temperature was held at 10° C. for another 2 hours. The solids were then recovered by filtration.

### Example 4

### Seeded Reactive Recrystallization Preparation of Form I of the Tert-Butylamine Salt of the Compound of Formula (I)

The compound of Formula (I) (12.8 wt %) in 80/20 methanol/ethyl acetate was prepared at 20° C. to form a crystallization mixture. The temperature was then ramped to 50° C. at 1° C./min and held for 75 minutes. Next, 1.5-1.6 molar equivalents of tert-butylamine was added the mixture to achieve a pH of 7.8 to 8.2. The mixture was then ramped to 60° C. at 1° C./min and held for 1 hour. The mixture was cooled to 50° C. at 0.3° C./min and held for at that temperature for 75 minutes. The mixture was seeded with 2 wt % seeds of Form I and then cooled to 10° C. at 0.08° C./min and held for 1 hour. Ethyl acetate was then added over a period of 2 hours until the ratio of methanol to ethyl acetate reached 20/80. The temperature was held at 10° C. for another 2 hours. The solids were then recovered by filtration.

### Example 5

### Preparation of Form II of the Tert-Butylamine Salt of the Compound of Formula (I)

The tert-butylamine salt of compound of Formula (I) was dissolved in 1,1,1,3,3,3-hexafluoroisopropyl alcohol (HFIPA) and allowed to evaporate slowly at ambient temperature to produce Form II of the tert-butylamine salt.

### Example 6

### Preparation of Crystalline Form III of the Tert-Butylamine Salt of the Compound of Formula

Crystal Form II of the tert-butylamine salt of compound of Formula (I) was heated between 77° C. and 90° C. and then further dried under vacuum at 40° C. overnight to Form III of the tert-butylamine salt.

### Example 7

### Preparation of Crystalline Form IV of the Tert-Butylamine Salt of the Compound of Formula

Crystal Form IV of the tert-butylamine salt of compound of Formula (I) was prepared according to the method of Example 5. Further slow evaporation provided Form IV.

### Example 8

### Crystalline Salt a and Crystalline Salt B Dissolution Profile

The dissolution profile of Crystalline Salt A and Crystalline Salt B were compared to the free acid of the compound of Formula (I).

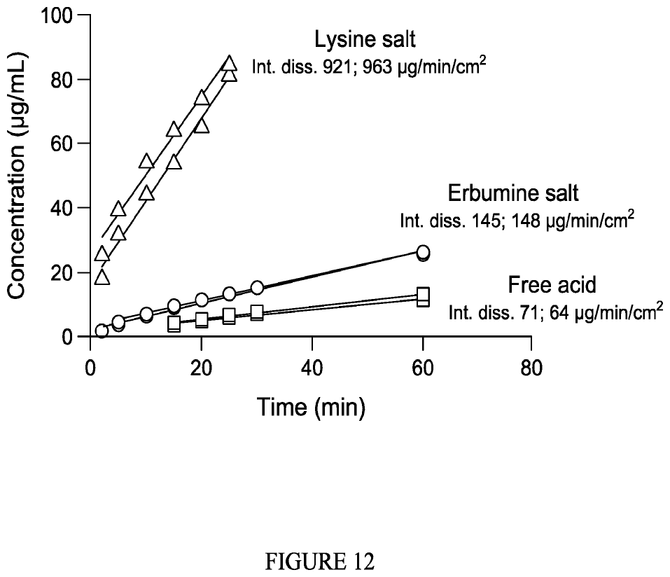

In a FaSSIF media (FIG. 12), both Crystalline Salt A and Crystalline Salt B demonstrated superior dissolution profiles compared to the solubility of the free acid of the compound of Formula (I).

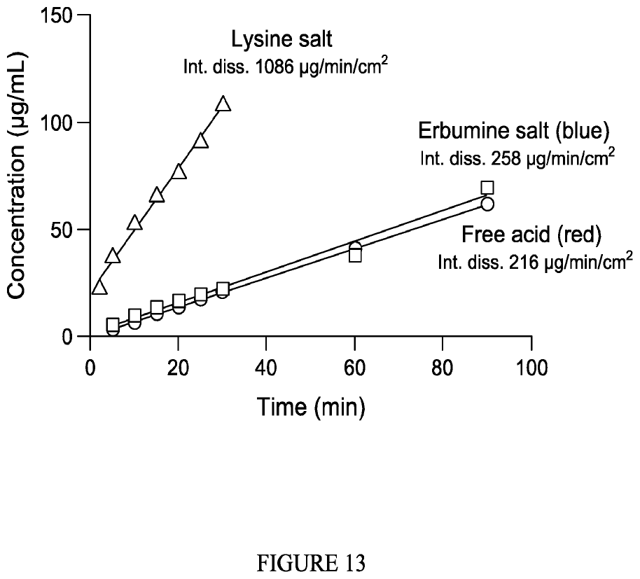

In a PBS media (FIG. 13), Crystalline Salt A demonstrated superior dissolution profile compared to the solubility of the free acid of the compound of Formula (I), while Crystalline Salt B demonstrated an equivalent, if not better, dissolution profile compared to the solubility of the free acid of the compound of Formula (I).

### Example 9

### Characterization of Crystalline Samples

The crystalline solid forms were characterized by XRPD, thermogravimetry (TG), and DTA.

The XRPD pattern of Crystalline Salt A (FIG. 1) shows a high degree of crystallinity. A melting temperature range at approximately 197-203° C. was observed using differential thermal analysis (FIG. 2). Crystalline Salt A showed a mass increase of 4.2 wt. % at 90% RH (1.3 equiv. of water) by dynamic vapor sorption/desorption analysis (FIG. 5).

The XRPD pattern of Crystalline Salt B Form I (FIG. 3) shows a high degree of crystallinity. A melting temperature range at approximately 196-224° C. was observed using differential thermal analysis (FIG. 4). Crystalline Salt B was non-hygroscopic by dynamic vapor sorption/desorption analysis (FIG. 6).

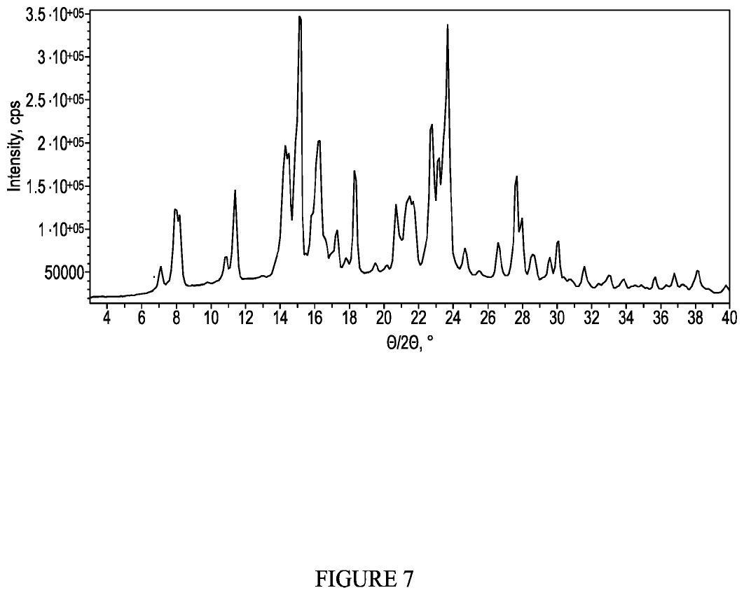

The XRPD pattern of Crystalline Salt B Form II (FIG. 7) shows a high degree of crystallinity.

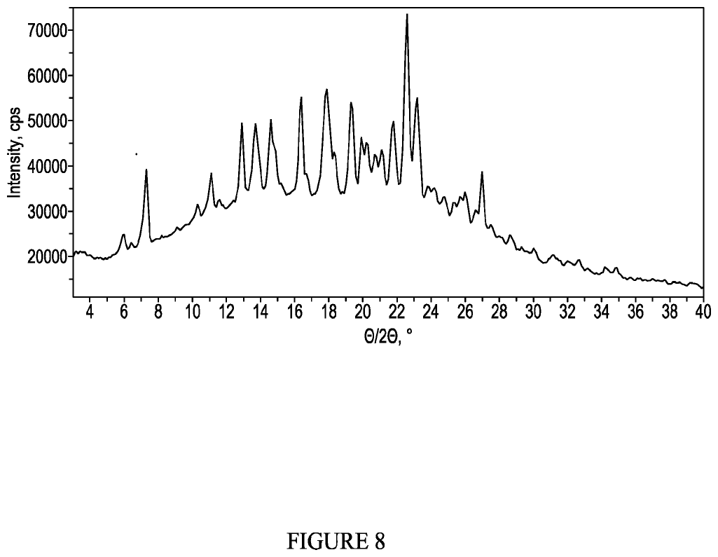

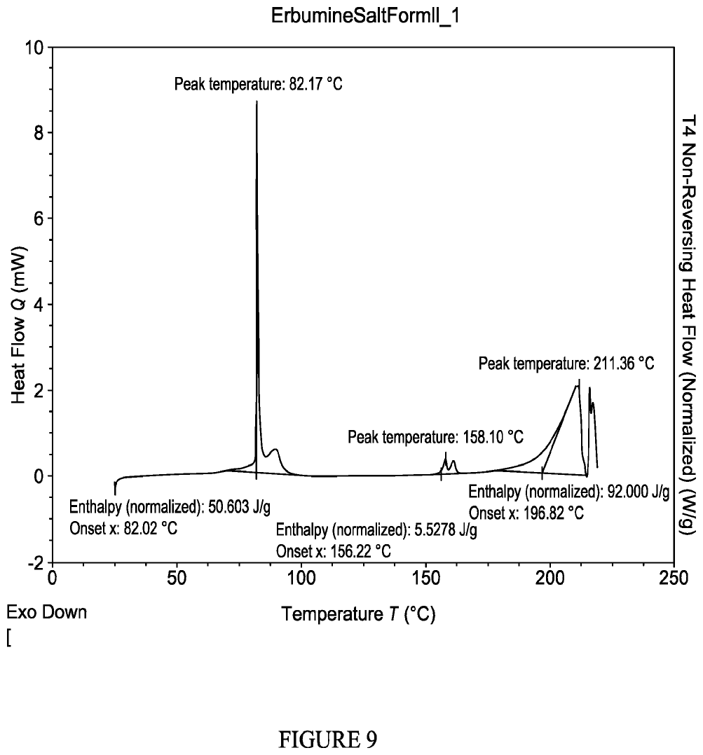

The XRPD pattern of Crystalline Salt B Form III (FIG. 8) shows a high degree of crystallinity. DSC data (FIG. 9) shows the loss of HFIPA around 80-90° C. followed by two endotherms at 155-165° C. and finally a large melting endotherm at ca. 200° C., consistent with Form I.

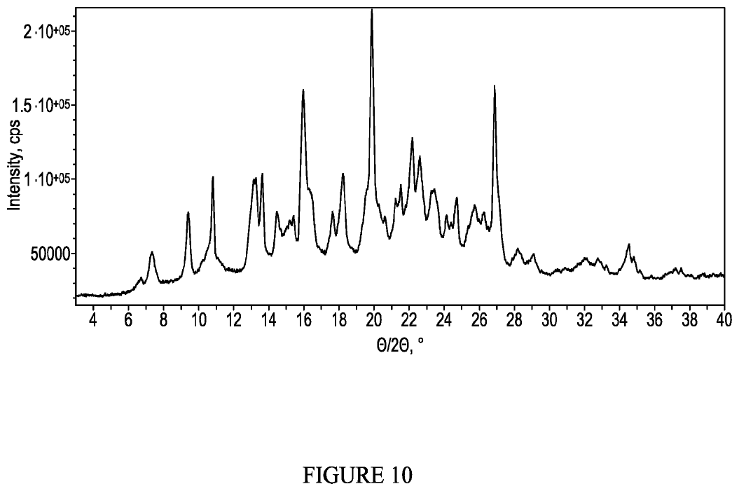

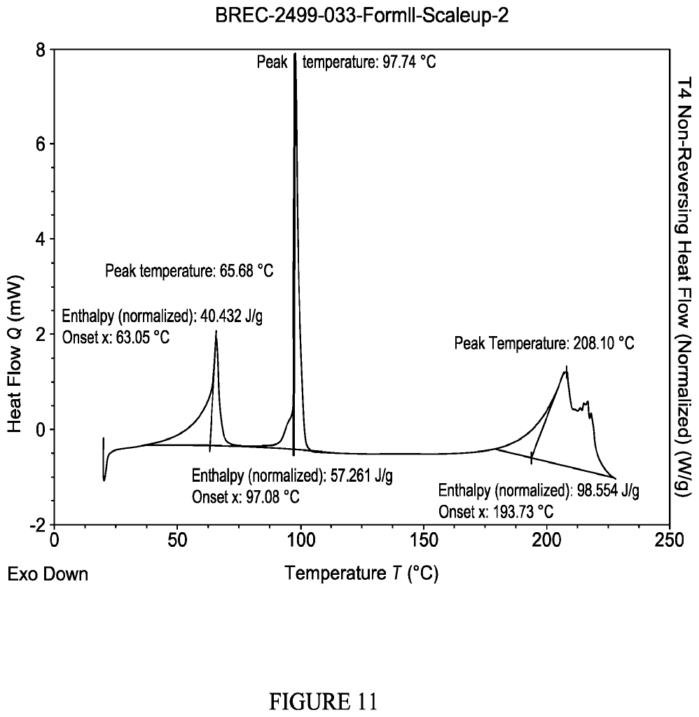

The XRPD pattern of Crystalline Salt B Form IV (FIG. 10) shows a high degree of crystallinity. DSC data (FIG. 11) shows that Form IV, on vacuum drying at 40° C., loses HFIPA and converts to Form III. This event at 80-100° C. is followed by two endotherms at 155-165° C. and finally a large melting endotherm at ca. 200° C., consistent with Form I.

### Example 10

### X-Ray Powder Diffraction (XRPD) Measurements of Crystalline Salt A

XRPD measurements of Crystalline Salt A of compound of Formula (I) were measured. Observed peaks are shown in Table 1. Prominent peaks are listed in Table 2. Note that none of the peaks are known to be representative or characteristic of this material since the state of preferred orientation in this sample is not known.

The range of data collected may be instrument dependent. Under most circumstances, it is sufficient for purposes of identify a polymorphic form by relying on a discrete number of XRPD peaks within the range of from 0° 2θ up to about 30° 2θ. Rounding algorithms were used to round each peak to the nearest 0.01° 2θ, based on the instrument used to collect the data and/or the inherent peak resolution. The location of the peaks along the x-axis (° 2θ) in both the figures and the tables were determined using proprietary software and rounded to one or two significant figures after the decimal point based upon the above criteria. Peak position variabilities are given to within ±0.2° 2θ. For d-space listings, the wavelength used to calculate the d-spacing was 1.54252 Å, (0.5:0.5, CuKα₁:CuKα₂).

TABLE 1
Observed Peaks for Crystalline Salt A

| Diffraction angle | | |
|---|---|---|
| °2θ (deg) | d space (Å) | Intensity (%) |
| 3.23 ± 0.20 | 27.317 | 3 |
| 4.23 ± 0.20 | 20.906 | 100 |
| 7.08 ± 0.20 | 12.490 | 2 |
| 7.92 ± 0.20 | 11.169 | 24 |
| 8.45 ± 0.20 | 10.459 | 14 |
| 10.59 ± 0.20 | 8.353 | 45 |
| 11.64 ± 0.20 | 7.606 | 8 |
| 11.98 ± 0.20 | 7.387 | 10 |
| 12.45 ± 0.20 | 7.112 | 1 |
| 12.71 ± 0.20 | 6.967 | 7 |
| 13.14 ± 0.20 | 6.736 | 3 |
| 13.69 ± 0.20 | 6.468 | 1 |
| 14.44 ± 0.20 | 6.136 | 24 |
| 15.25 ± 0.20 | 5.809 | 40 |
| 15.50 ± 0.20 | 5.716 | 29 |
| 15.85 ± 0.20 | 5.593 | 8 |
| 16.65 ± 0.20 | 5.324 | 17 |
| 17.23 ± 0.20 | 5.147 | 1 |
| 17.66 ± 0.20 | 5.023 | 20 |
| 18.03 ± 0.20 | 4.920 | 7 |
| 18.33 ± 0.20 | 4.839 | 12 |
| 18.71 ± 0.20 | 4.743 | 13 |
| 19.32 ± 0.20 | 4.594 | 35 |
| 19.87 ± 0.20 | 4.469 | 29 |
| 20.37 ± 0.20 | 4.361 | 19 |
| 20.94 ± 0.20 | 4.243 | 8 |
| 21.30 ± 0.20 | 4.171 | 26 |
| 21.84 ± 0.20 | 4.069 | 5 |
| 22.63 ± 0.20 | 3.929 | 24 |
| 23.41 ± 0.20 | 3.801 | 36 |
| 23.94 ± 0.20 | 3.717 | 62 |
| 24.42 ± 0.20 | 3.645 | 25 |
| 25.30 ± 0.20 | 3.520 | 62 |
| 25.81 ± 0.20 | 3.452 | 46 |
| 26.00 ± 0.20 | 3.427 | 31 |
| 26.40 ± 0.20 | 3.376 | 12 |
| 26.75 ± 0.20 | 3.333 | 7 |
| 27.26 ± 0.20 | 3.272 | 2 |
| 28.10 ± 0.20 | 3.176 | 13 |
| 28.91 ± 0.20 | 3.088 | 4 |
| 29.77 ± 0.20 | 3.001 | 5 |

Table 2 provides XRPD data identified as “prominent peaks,” as that terms is used here. Prominent peaks are a subset of the entire observed peak list. Prominent peaks are selected from observed peaks by identifying preferably non-overlapping, low-angle peaks, with strong intensity.

TABLE 2
Prominent Peaks for Crystalline Salt A

| Diffraction angle | | |
|---|---|---|
| °2θ (deg) | d space (Å) | Intensity (%) |
| 4.23 ± 0.20 | 20.906 | 100 |
| 7.92 ± 0.20 | 11.169 | 24 |
| 10.59 ± 0.20 | 8.353 | 45 |
| 14.44 ± 0.20 | 6.136 | 24 |
| 15.25 ± 0.20 | 5.809 | 40 |
| 15.50 ± 0.20 | 5.716 | 29 |
| 19.32 ± 0.20 | 4.594 | 35 |
| 19.87 ± 0.20 | 4.469 | 29 |
| 21.30 ± 0.20 | 4.171 | 26 |
| 23.41 ± 0.20 | 3.801 | 36 |
| 23.94 ± 0.20 | 3.717 | 62 |
| 24.42 ± 0.20 | 3.645 | 25 |
| 25.30 ± 0.20 | 3.520 | 62 |
| 25.81 ± 0.20 | 3.452 | 46 |
| 26.00 ± 0.20 | 3.427 | 31 |

### Example 11

### X-Ray Powder Diffraction (XRPD) Measurements of Crystalline Salt B Form I

XRPD measurements of Form I of Crystalline Salt B of compound of Formula (I) were measured. Observed peaks are shown in Table 3. Prominent peaks are listed in Table 4. Note that none of the peaks are known to be representative or characteristic of this material since the state of preferred orientation in this sample is not known.

The range of data collected may be instrument dependent. Under most circumstances, it is sufficient for purposes of identify a polymorphic form by relying on a discrete number of XRPD within the range of from 0° 2θ up to about 30° 2θ were selected. Rounding algorithms were used to round each peak to the nearest 0.01° 2θ, based on the instrument used to collect the data and/or the inherent peak resolution. The location of the peaks along the x-axis (° 2θ) in both the figures and the tables were determined using proprietary software and rounded to one or two significant figures after the decimal point based upon the above criteria. Peak position variabilities are given to within ±0.2° 2θ. For d-space listings, the wavelength used to calculate d-spacings was 1.54252 Å, the weighted average of the Cu-Kα₁ and Cu-Kα₂ wavelength.

TABLE 3
Observed Peaks for Crystalline Salt B Form I

| Diffraction angle | | |
|---|---|---|
| °2θ (deg) | d space (Å) | Intensity (%) |
| 6.01 ± 0.20 | 14.712 | 3 |
| 7.76 ± 0.20 | 11.391 | 0 |
| 8.17 ± 0.20 | 10.818 | 0 |
| 9.03 ± 0.20 | 9.789 | 0 |
| 9.49 ± 0.20 | 9.317 | 0 |
| 10.08 ± 0.20 | 8.780 | 1 |
| 10.69 ± 0.20 | 8.278 | 1 |
| 11.61 ± 0.20 | 7.622 | 3 |
| 12.38 ± 0.20 | 7.151 | 2 |
| 13.19 ± 0.20 | 6.714 | 9 |
| 13.59 ± 0.20 | 6.515 | 19 |
| 13.75 ± 0.20 | 6.443 | 93 |
| 15.61 ± 0.20 | 5.677 | 1 |
| 16.03 ± 0.20 | 5.527 | 0 |
| 16.81 ± 0.20 | 5.276 | 0 |
| 17.65 ± 0.20 | 5.024 | 17 |
| 17.93 ± 0.20 | 4.948 | 42 |
| 18.36 ± 0.20 | 4.829 | 75 |
| 18.44 ± 0.20 | 4.820 | 44 |
| 19.39 ± 0.20 | 4.574 | 60 |
| 20.26 ± 0.20 | 4.379 | 47 |
| 20.66 ± 0.20 | 4.296 | 9 |
| 20.77 ± 0.20 | 4.274 | 35 |
| 21.22 ± 0.20 | 4.183 | 100 |
| 21.78 ± 0.20 | 4.078 | 27 |
| 21.84 ± 0.20 | 4.076 | 28 |
| 22.47 ± 0.20 | 3.954 | 2 |
| 22.82 ± 0.20 | 3.894 | 38 |
| 23.09 ± 0.20 | 3.848 | 4 |
| 23.34 ± 0.20 | 3.809 | 19 |
| 23.91 ± 0.20 | 3.719 | 18 |
| 24.47 ± 0.20 | 3.635 | 3 |
| 24.87 ± 0.20 | 3.578 | 12 |
| 25.41 ± 0.20 | 3.502 | 6 |
| 26.12 ± 0.20 | 3.409 | 20 |
| 26.49 ± 0.20 | 3.361 | 14 |
| 26.85 ± 0.20 | 3.318 | 4 |
| 27.49 ± 0.20 | 3.242 | 16 |
| 27.72 ± 0.20 | 3.216 | 5 |
| 28.18 ± 0.20 | 3.164 | 3 |
| 28.50 ± 0.20 | 3.130 | 1 |
| 28.78 ± 0.20 | 3.099 | 8 |
| 29.30 ± 0.20 | 3.045 | 6 |
| 29.78 ± 0.20 | 2.998 | 1 |

Table 4 provides XRPD data identified as “prominent peaks.” Prominent peaks are a subset of the entire observed peak list. Prominent peaks are selected from observed peaks by identifying preferably non-overlapping, low-angle peaks, with strong intensity.

TABLE 4
Prominent Peaks for Crystalline Salt B Form I

| Diffraction angle | | |
|---|---|---|
| °2θ (deg) | d space (Å) | Intensity (%) |
| 13.59 ± 0.20 | 6.515 | 19 |
| 13.75 ± 0.20 | 6.443 | 93 |
| 17.93 ± 0.20 | 4.948 | 42 |
| 18.36 ± 0.20 | 4.829 | 75 |
| 18.44 ± 0.20 | 4.820 | 44 |
| 19.39 ± 0.20 | 4.574 | 60 |
| 20.26 ± 0.20 | 4.379 | 47 |
| 20.77 ± 0.20 | 4.274 | 35 |
| 21.22 ± 0.20 | 4.183 | 100 |
| 21.78 ± 0.20 | 4.078 | 27 |
| 21.84 ± 0.20 | 4.076 | 28 |
| 22.82 ± 0.20 | 3.894 | 38 |
| 23.34 ± 0.20 | 3.809 | 19 |
| 23.91 ± 0.20 | 3.719 | 18 |
| 26.12 ± 0.20 | 3.409 | 20 |

### Example 12

### X-Ray Powder Diffraction (XRPD) Measurements of Crystalline Salt B Form II

XRPD measurements of Form II of Crystalline Salt B of compound of Formula (I) were measured. Observed peaks are shown in Table 5. Prominent peaks are listed in Table 6. Note that none of the peaks are known to be representative or characteristic of this material since the state of preferred orientation in this sample is not known.

The range of data collected may be instrument dependent. Under most circumstances, it is sufficient for purposes of identify a polymorphic form by relying on a discrete number of XRPD within the range of from 0° 2θ up to about 30° 2θ were selected. Rounding algorithms were used to round each peak to the nearest 0.01° 2θ, based on the instrument used to collect the data and/or the inherent peak resolution. The location of the peaks along the x-axis (° 2θ) in both the figures and the tables were determined using proprietary software and rounded to one or two significant figures after the decimal point based upon the above criteria. Peak position variabilities are given to within ±0.2° 2θ. For d-space listings, the wavelength used to calculate d-spacings was 1.54252 Å, the weighted average of the Cu-Kα₁ and Cu-Kα₂ wavelength.

TABLE 5
Observed Peaks for Crystalline Salt B Form II

| Diffraction angle | |
|---|---|
| °2θ (deg) | Intensity (%) |
| 7.08 ± 0.20 | 9 |
| 8.01 ± 0.20 | 45 |
| 10.84 ± 0.20 | 7 |
| 11.39 ± 0.20 | 35 |
| 14.33 ± 0.20 | 66 |
| 15.12 ± 0.20 | 100 |
| 16.22 ± 0.20 | 56 |
| 17.27 ± 0.20 | 13 |
| 17.80 ± 0.20 | 3 |
| 18.34 ± 0.20 | 40 |
| 20.73 ± 0.20 | 29 |
| 21.44 ± 0.20 | 79 |
| 22.74 ± 0.20 | 37 |
| 23.70 ± 0.20 | 65 |
| 24.70 ± 0.20 | 11 |
| 26.63 ± 0.20 | 17 |
| 27.66 ± 0.20 | 37 |
| 27.96 ± 0.20 | 34 |
| 28.59 ± 0.20 | 20 |
| 29.59 ± 0.20 | 8 |

Table 6 provides XRPD data identified as “prominent peaks.” Prominent peaks are a subset of the entire observed peak list. Prominent peaks are selected from observed peaks by identifying preferably non-overlapping, low-angle peaks, with strong intensity.

TABLE 6
Prominent Peaks for Crystalline Salt B Form II

| Diffraction angle | |
|---|---|
| °2θ (deg) | Intensity (%) |
| 8.01 ± 0.20 | 45 |
| 11.39 ± 0.20 | 35 |
| 14.33 ± 0.20 | 66 |
| 15.12 ± 0.20 | 100 |
| 16.22 ± 0.20 | 56 |
| 18.34 ± 0.20 | 40 |
| 21.44 ± 0.20 | 79 |
| 22.74 ± 0.20 | 37 |
| 23.70 ± 0.20 | 65 |
| 27.66 ± 0.20 | 37 |

### Example 13

### X-Ray Powder Diffraction (XRPD) Measurements of Crystalline Salt B Form III

XRPD measurements of Form III of Crystalline Salt B of compound of Formula (I) were measured. Observed peaks are shown in Table 7. Prominent peaks are listed in Table 8. Note that none of the peaks are known to be representative or characteristic of this material since the state of preferred orientation in this sample is not known.

The range of data collected may be instrument dependent. Under most circumstances, it is sufficient for purposes of identify a polymorphic form by relying on a discrete number of XRPD within the range of from 0° 2θ up to about 30° 2θ were selected. Rounding algorithms were used to round each peak to the nearest 0.01° 2θ, based on the instrument used to collect the data and/or the inherent peak resolution. The location of the peaks along the x-axis (° 2θ) in both the figures and the tables were determined using proprietary software and rounded to one or two significant figures after the decimal point based upon the above criteria. Peak position variabilities are given to within ±0.2° 2θ. For d-space listings, the wavelength used to calculate d-spacings was 1.54252 Å, the weighted average of the Cu-Kα₁ and Cu-Kα₂ wavelength.

TABLE 7
Observed Peaks for Crystalline Salt B Form III

| Diffraction angle | |
|---|---|
| °2θ (deg) | Intensity (%) |
| 5.93 ± 0.20 | 9 |
| 7.27 ± 0.20 | 34 |
| 11.09 ± 0.20 | 20 |
| 12.90 ± 0.20 | 35 |
| 13.72 ± 0.20 | 55 |
| 14.64 ± 0.20 | 72 |
| 16.37 ± 0.20 | 50 |
| 17.87 ± 0.20 | 100 |
| 19.33 ± 0.20 | 70 |
| 20.02 ± 0.20 | 48 |
| 21.78 ± 0.20 | 45 |
| 22.57 ± 0.20 | 89 |
| 23.18 ± 0.20 | 55 |
| 25.82 ± 0.20 | 25 |
| 27.00 ± 0.20 | 22 |

Table 8 provides XRPD data identified as “prominent peaks.” Prominent peaks are a subset of the entire observed peak list. Prominent peaks are selected from observed peaks by identifying preferably non-overlapping, low-angle peaks, with strong intensity.

TABLE 8
Prominent Peaks for Crystalline Salt B Form III

| Diffraction angle | |
|---|---|
| °2θ (deg) | Intensity (%) |
| 12.90 ± 0.20 | 35 |
| 13.72 ± 0.20 | 55 |
| 14.64 ± 0.20 | 72 |
| 16.37 ± 0.20 | 50 |
| 17.87 ± 0.20 | 100 |
| 19.33 ± 0.20 | 70 |
| 20.02 ± 0.20 | 48 |
| 21.78 ± 0.20 | 45 |
| 22.57 ± 0.20 | 89 |
| 23.18 ± 0.20 | 55 |

### Example 14

### X-Ray Powder Diffraction (XRPD) Measurements of Crystalline Salt B Form IV

XRPD measurements of Form IV of Crystalline Salt B of compound of Formula (I) were measured. Observed peaks are shown in Table 9. Prominent peaks are listed in Table 10. Note that none of the peaks are known to be representative or characteristic of this material since the state of preferred orientation in this sample is not known.

The range of data collected may be instrument dependent. Under most circumstances, it is sufficient for purposes of identify a polymorphic form by relying on a discrete number of XRPD within the range of from 0° 2θ up to about 30° 2θ were selected. Rounding algorithms were used to round each peak to the nearest 0.01° 2θ, based on the instrument used to collect the data and/or the inherent peak resolution. The location of the peaks along the x-axis (° 2θ) in both the figures and the tables were determined using proprietary software and rounded to one or two significant figures after the decimal point based upon the above criteria. Peak position variabilities are given to within ±0.2° 2θ. For d-space listings, the wavelength used to calculate d-spacings was 1.54252 Å, the weighted average of the Cu-Kα₁ and Cu-Kα₂ wavelength.

TABLE 9
Observed Peaks for Crystalline Salt B Form IV

| Diffraction angle | |
|---|---|
| °2θ (deg) | Intensity (%) |
| 7.35 ± 0.20 | 27 |
| 9.41 ± 0.20 | 35 |
| 10.81 ± 0.20 | 25 |
| 13.16 ± 0.20 | 100 |
| 13.63 ± 0.20 | 32 |
| 14.48 ± 0.20 | 17 |
| 15.35 ± 0.20 | 13 |
| 15.96 ± 0.20 | 69 |
| 17.63 ± 0.20 | 15 |
| 18.22 ± 0.20 | 44 |
| 19.88 ± 0.20 | 90 |
| 21.22 ± 0.20 | 6 |
| 21.53 ± 0.20 | 7 |
| 22.16 ± 0.20 | 28 |
| 22.61 ± 0.20 | 70 |
| 23.39 ± 0.20 | 33 |
| 24.70 ± 0.20 | 20 |
| 25.68 ± 0.20 | 21 |
| 26.91 ± 0.20 | 78 |
| 28.22 ± 0.20 | 13 |
| 29.09 ± 0.20 | 10 |

Table 10 provides XRPD data identified as “prominent peaks.” Prominent peaks are a subset of the entire observed peak list. Prominent peaks are selected from observed peaks by identifying preferably non-overlapping, low-angle peaks, with strong intensity.

TABLE 10
Prominent Peaks for Crystalline Salt B Form IV

| Diffraction angle | |
|---|---|
| °2θ (deg) | Intensity (%) |
| 9.41 ± 0.20 | 35 |
| 13.16 ± 0.20 | 100 |
| 13.63 ± 0.20 | 32 |
| 15.96 ± 0.20 | 69 |
| 18.22 ± 0.20 | 44 |
| 19.88 ± 0.20 | 90 |
| 22.61 ± 0.20 | 70 |
| 23.39 ± 0.20 | 33 |
| 26.91 ± 0.20 | 78 |

### Example 15

### X-Ray Powder Diffraction (XRPD) Measurements of Alternative Crystalline Salts

XRPD measurements of a Crystalline Salt form of compound of Formula (I) are measured. Peaks are observed, but none of the peaks are known to be representative or characteristic of this material since the state of preferred orientation in this sample is unknown.

The range of data collected may be instrument dependent. Under most circumstances, it is sufficient for purposes of identify a polymorphic form by relying on a discrete number of XRPD peaks within the range of from 0° 2θ up to about 30° 2θ. Rounding algorithms were used to round each peak to the nearest 0.01° 2θ, based on the instrument used to collect the data and/or the inherent peak resolution. The location of the peaks along the x-axis (° 2θ) in both the figures and the tables were determined using proprietary software and rounded to one or two significant figures after the decimal point based upon the above criteria. Peak position variabilities are given to within ±0.2° 2θ. For d-space listings, the wavelength used to calculate d-spacings was 1.54252 Å, the weighted average of the Cu-Kα₁ and Cu-Kα₂ wavelength.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the application.

What is claimed is:

1. A crystalline form of a pharmaceutically acceptable salt of the compound of Formula (I):

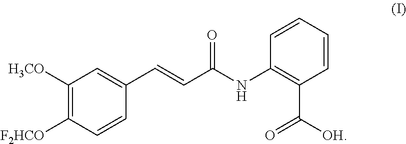 [SMILES: COc1cc(/C=C/C(=O)Nc2ccccc2C(=O)O)ccc1OC(F)F]

2. The crystalline form of claim 1, wherein the pharmaceutically acceptable salt is selected from the group consisting of: a sodium salt, a potassium salt, a zinc salt, a magnesium salt, and a calcium salt.

3. The crystalline form of claim 1, wherein the pharmaceutically acceptable salt is selected from the group consisting of: a choline salt, an L-lysine salt, a tert-butylamine salt, and a diethanolamine salt.

4. The crystalline form of claim 3, wherein the pharmaceutically acceptable salt is an L-lysine salt.

5. The crystalline form of claim 4, wherein the crystalline form exhibits an X-ray powder diffraction pattern having at least three characteristic peaks, wherein the three characteristic peaks are selected from the group consisting of peaks at 4.23, 7.92, 10.59, 14.44, 15.25, 15.50, 19.32, 19.87, 21.30, 23.41, 23.94, 24.42, 25.30, 25.81, 26.00 degrees 2θ (±0.2 degrees 2θ).

6. The crystalline form of claim 4, wherein the crystalline form exhibits an X-ray powder diffraction pattern having at least four characteristic peaks, wherein the four characteristic peaks are selected from the group consisting of peaks at 4.23, 7.92, 10.59, 14.44, 15.25, 15.50, 19.32, 19.87, 21.30, 23.41, 23.94, 24.42, 25.30, 25.81, 26.00 degrees 2θ (±0.2 degrees 2θ).

7. The crystalline form of claim 4, wherein the crystalline form exhibits an X-ray powder diffraction pattern having at least five characteristic peaks, wherein the five characteristic peaks are selected from the group consisting of peaks at 4.23, 7.92, 10.59, 14.44, 15.25, 15.50, 19.32, 19.87, 21.30, 23.41, 23.94, 24.42, 25.30, 25.81, 26.00 degrees 2θ (±0.2 degrees 2θ).

8. The crystalline form of claim 4, wherein the crystalline form exhibits an X-ray powder diffraction pattern as depicted in FIG. 1.

9. The crystalline form of any one of claims 4 to 8, wherein the crystalline form has a melting point of about 197-203° C.

10. The crystalline form of claim 3, wherein the pharmaceutically acceptable salt is a tert-butylamine salt.

11. The crystalline form of claim 10, wherein the crystalline form exhibits an X-ray powder diffraction pattern having at least three characteristic peaks, wherein the three characteristic peaks are selected from the group consisting of peaks at 13.59, 13.75, 17.93, 18.36, 18.44, 19.39, 20.26, 20.77, 21.22, 21.78, 21.84, 22.82, 23.34, 23.91, 26.12 degrees 2θ (±0.2 degrees 2θ).

12. The crystalline form of claim 10, wherein the crystalline form exhibits an X-ray powder diffraction pattern having at least four characteristic peaks, wherein the four characteristic peaks are selected from the group consisting of peaks at 13.59, 13.75, 17.93, 18.36, 18.44, 19.39, 20.26, 20.77, 21.22, 21.78, 21.84, 22.82, 23.34, 23.91, 26.12 degrees 2θ (±0.2 degrees 2θ).

13. The crystalline form of claim 10, wherein the crystalline form exhibits an X-ray powder diffraction pattern having at least five characteristic peaks, wherein the five characteristic peaks are selected from the group consisting of peaks at 13.59, 13.75, 17.93, 18.36, 18.44, 19.39, 20.26, 20.77, 21.22, 21.78, 21.84, 22.82, 23.34, 23.91, 26.12 degrees 2θ (±0.2 degrees 2θ).

14. The crystalline form of claim 10, wherein the crystalline form exhibits an X-ray powder diffraction pattern as depicted in FIG. 3.

15. The crystalline form of claim 10 to 14, wherein the crystalline form has a melting point of about 196-224° C.

16. A pharmaceutical composition comprising a crystalline form of any one of claims 1 to 16 and a pharmaceutically acceptable excipient.

17. The L-lysine salt of the compound of Formula (I):

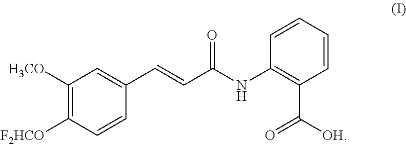 [SMILES: COc1cc(/C=C/C(=O)Nc2ccccc2C(=O)O)ccc1OC(F)F]

18. The tert-butylamine salt of the compound of Formula (I):

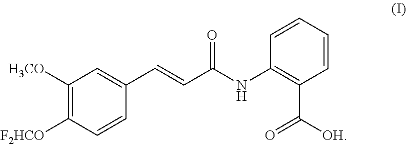 [SMILES: COc1cc(/C=C/C(=O)Nc2ccccc2C(=O)O)ccc1OC(F)F]

19. A method of treating an eye disease comprising administering to a subject a therapeutically effective amount of the crystalline form of any one of claims 1 to 16.

20. The method of claim 19, wherein the eye disease selected from the group comprising diabetic retinopathy, proliferative vitreoretinopathy corneal edema, anterior and posterior uveitis, pterygium, corneal diseases, dry eye, conjunctivitis, allergy- and laser-induced exudation, non-age related macular degeneration, macular edema, age-related macular degeneration and ocular von Hippel-Lindau disease.

21. A method of reducing at least one of: the level of ICAM-1 mRNA in the retina of the subject; the level of VEGF mRNA in the retina of the subject; and the number of leukocytes in the retina of the subject, the method comprising administering to a subject a therapeutically effective amount of the crystalline form of any one of claims 1 to 15 or the pharmaceutical composition of claim 16.

22. A method of treating a disease or condition associated with fibrosis, the method comprising administering to a subject a therapeutically effective amount of the crystalline form of any one of claims 1 to 15 or the pharmaceutical composition of claim 16.

23. The method of claim 22, wherein the disease or disorder associated with fibrosis is selected from the group consisting of: a fibrotic skin disorder, a lung disease, a heart disease, and a kidney disease.

24. The method of claim 23, wherein the fibrotic skin disorder is selected from the group consisting of: keloids, hypertrophic scars and scleroderma.

25. The method of claim 23, wherein the lung disease is pulmonary fibrosis.

26. The method of claim 23, wherein the heart disease is selected from the group consisting of: heart failure due to ischaemic heart disease, diabetic heart disease, valvular heart disease, hypertensive heart disease, diabetic cardiomyopathy, and hypertension.

27. The method of claim 23, wherein the kidney disease is selected from the group consisting of: progressive glomerular kidney disease and diabetic kidney disease.

28. The method of claim 27, wherein the progressive kidney disease is primary glomerulonephritis or secondary glomerulonephritis.

29. The method of claim 28, wherein the primary glomerulonephritis is selected from the group consisting of: membranous nephropathy, focal segmental glomerulosclerosis, membranoproliferative glomerulonephritis, diffuse proliferative glomerulonephritis, and membranous focal segmental glomerulosclerosis.

30. The method of claim 28, wherein the secondary glomerulonephritis is diabetic nephropathy or ischemic nephropathy.

31. The method of claim 23, wherein the kidney disease is a progressive kidney diseases with origins primarily in the tubulointerstitium.

32. The method of claim 31, wherein the kidney disease is interstitial nephritis, autosomal dominant tubulointerstitial fibrosis, or reflux nephropathy.

33. The method of any one of claims 19 to 32, wherein the crystalline form or composition is administered orally.

34. The method of any one of claims 19 to 32, wherein the crystalline form or composition is administered to the eye.

35. A method of treating an eye disease comprising administering to a subject a therapeutically effective amount of the compound of any one of claims 17 and 18.

36. The method of claim 35, wherein the eye disease selected from the group comprising diabetic retinopathy, proliferative vitreoretinopathy corneal edema, anterior and posterior uveitis, pterygium, corneal diseases, dry eye, conjunctivitis, allergy- and laser-induced exudation, non-age related macular degeneration, macular edema, age-related macular degeneration and ocular von Hippel-Lindau disease.

37. A method of reducing at least one of: the level of ICAM-1 mRNA in the retina of the subject; the level of VEGF mRNA in the retina of the subject; and the number of leukocytes in the retina of the subject, the method comprising administering to a subject a therapeutically effective amount of the compound of any one of claim 17 or 18.

38. A method of treating a disease or condition associated with fibrosis, the method comprising administering to a subject a therapeutically effective amount of the compound of claim 17 or 18.

39. The method of claim 38, wherein the disease or disorder associated with fibrosis is selected from the group consisting of: a fibrotic skin disorder, a lung disease, a heart disease, and a kidney disease.

40. The method of claim 39, wherein the fibrotic skin disorder is selected from the group consisting of: keloids, hypertrophic scars and scleroderma.

41. The method of claim 39, wherein the lung disease is pulmonary fibrosis.

42. The method of claim 39, wherein the heart disease is selected from the group consisting of: heart failure due to ischaemic heart disease, diabetic heart disease, valvular heart disease, hypertensive heart disease, diabetic cardiomyopathy, and hypertension.

43. The method of claim 39, wherein the kidney disease is selected from the group consisting of: progressive glomerular kidney disease and diabetic kidney disease.

44. The method of claim 43, the progressive glomerular kidney disease is primary glomerulonephritis or secondary glomerulonephritis.

45. The method of claim 44, wherein the primary glomerulonephritis is selected from the group consisting of: membranous nephropathy, focal segmental glomerulosclerosis, membranoproliferative glomerulonephritis, diffuse proliferative glomerulonephritis, and membranous focal segmental glomerulosclerosis.

46. The method of claim 44, wherein the secondary glomerulonephritis is diabetic nephropathy or ischemic nephropathy.

47. The method of claim 39, wherein the kidney disease is a progressive kidney diseases with origins primarily in the tubulointerstitium.

48. The method of claim 47, wherein the kidney disease is interstitial nephritis, autosomal dominant tubulointerstitial fibrosis, or reflux nephropathy.

49. The method of any one of claims 35 to 48, wherein the compound is administered orally.

50. The method of any one of claims 35 to 48, wherein the compound is administered to the eye.
